(19) United States
(12) Patent Application Publication
(10) Pub. No.: US 2006/0073095 A1
(43) Pub. Date: Apr. 6, 2006

(54) Methods for screening antibody-producing cells on heterogeneous antigen substrates

(76) Inventor: Steven Kessler, Belmont, CA (US)

Correspondence Address: FLIESLER MEYER, LLP, FOUR EMBARCADERO CENTER, SUITE 400, SAN FRANCISCO, CA, 94111, US

(21) Appl. No.: 10/224,997

(22) Filed: Aug. 21, 2002

Related U.S. Application Data
(60) Provisional application No. 60/314,070, filed on Aug. 22, 2001.
(60) Provisional application No. 60/314,071, filed on Aug. 22, 2001.

(51) Int. Cl. A61K 51/00; C12P 21/04; C12N 5/06

(52) U.S. Cl. 424/1.11; 435/70.21; 435/326; 530/387.1

(57) ABSTRACT

Methods and compounds are disclosed that relate to screening and selection of monoclonal antibodies specific for antigens in heterogeneous antigen mixtures. Antibody-secreting cells such as hybridomas are modified to make them capable of directly binding antigens by capturing their secreted antibody products onto their surface membranes in appropriate binding density and orientation. Selectivity of binding to novel or desired antigens is achieved by first reacting the antigen mixtures affixed to a solid substrate with a polyclonal antibody library that prevents access to the majority of antigens or epitopes other than those that are novel or desired.

### RELATED APPLICATIONS

This Application claims priority under 35 U.S.C. §119 to U.S. Provisional Patent Applications Ser. No. 60/314,070 filed Aug. 22, 2001 and Ser. No. 60/314,071 filed Aug. 22, 2001 and is related to United States Utility Patent Application titled “Methods for Screening Monoclonal Antibodies on Heterogeneous Antigen Substrates”, Steven Kessler, inventor, Attorney Docket No: KSLR 1000 US1 SRM/DBB, filed concurrently. Each of the above-identified applications is herein incorporated fully by reference.

### BACKGROUND OF THE INVENTION

1. Field of the Invention

This invention relates to the screening and production of monoclonal antibodies and phage antibodies for binding to a molecular target. Specifically this invention relates to using a polyclonal antibody library directed against a number of undesirable antigens to mask those antigens so that antibody-secreting cells directed toward desirable antigens can adhere to those antigens on a substrate and can be more effectively selected.

2. Description of Related Art

The value of monoclonal antibodies as therapeutic immune effector and drug delivery agents, as tools for in vitro disease diagnosis and in vivo disease imaging, and as tools for discovery of new drugs continues to rise. Early clinical studies have shown monoclonal antibodies to produce durable responses in several solid cancers, and partial alleviation of symptoms in certain autoimmune and inflammatory diseases. Used as diagnostic and imaging tools, they can prove invaluable for staging malignancies, for monitoring the progression of disease and responses to therapy, and for the development of more patient-specific therapies. Used in conjunction with genomics-based and proteomics-based or protein biochip array approaches, monoclonal antibodies can play key roles in discovery of molecular pathways and drug targets. In view of these applications there is much interest in the continuing discovery of monoclonal antibodies to additional antigenic biomarkers, and especially on the surfaces of cells associated with disease processes.

Monoclonal antibodies are antibodies obtained from a population of substantially homogeneous antibodies. The individual molecules comprising the population are identical except for possible naturally occurring mutations that can be present in minor amounts. Monoclonal antibodies are highly specific, being directed against a single antigenic determinant (epitope). In contrast to conventional (polyclonal or oligoclonal) antibody preparations which typically include different antibodies directed against different antigens or determinants, each monoclonal antibody is directed against a single determinant on the antigen.

The modifier “monoclonal” indicates the character of the antibody as being obtained from a substantially homogeneous population of antibodies, and is not to be construed as requiring production of the antibody by any particular method. For example, the monoclonal antibodies to be used in an embodiment of the present invention can be made by the hybridoma method first described by Kohler et al., Nature 256:495 (1975) or its subsequent modifications, or can be made by recombinant DNA methods. Monoclonal antibodies can also be isolated from phage antibody libraries generated using methods such as those described by McCafferty and Johnson (in: Phage Display of Peptides and Proteins; Kay, Winter and McCafferty, eds., Academic Press 1996, pp. 79-111). In addition, monoclonal antibodies can be made by extracting the mRNAs or genomic DNAs encoding the specific antibody chains produced by hybridoma cells or antibody-forming or plaque-forming cells, and using this material to clone and express the encoding cDNAs in other cells.

Human myeloma (Karpas A, et al., Proc Natl Acad Sci USA (2001) 98:1799) and mouse-human heteromyeloma (Arinbjarnarson S and Valdimarsson II (2002) J Immunol Methods (2002) 259:139) cell lines also have been described for the production of human monoclonal antibodies. A rabbit myeloma cell line has been described for producing rabbit monoclonal antibodies (Spieker-Polet H, et al., Proc Natl Acad Sci USA (1995) 92: 9348). It can be appreciated that any type of myeloma or related cell lines that can fuse efficiently with lymphocytes can be advantageously with the methods and compositions of this invention.

In standard hybridoma methods, a mouse or other appropriate animal, such as a hamster, rat, rabbit, cow, sheep, monkey or human, and the like is immunized with antigen to elicit lymphocytes that produce or are capable of producing antibodies that will specifically bind to the molecules used for immunization. These antibodies may be immunoglobulin molecules native to the species of origin, or they may be human antibodies produced by transgenic human immunoglobulin-producing animals such as mice, rabbits or cows (e.g., Abgenix, Fremont, Calif.; Medarex, Princeton, N.J.; Hematech, Westport, Conn.).

The hybridoma cells thus obtained are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Seeding of the hybridoma cells is generally performed at a suitably low cell density, most commonly in 96-well microtiter plates or trays, so that after drug selection each well preferably contains no more than one surviving hybridoma clone. The culture medium or supernatant from each well in which hybridoma cells are growing is then assayed or screened individually for production of monoclonal antibodies directed against the antigen. This typically requires the collection and screening of culture medium from hundreds to several thousands or more of hybridomas produced from the lymphoid tissues of a single immunized host animal.

Also, the binding specificity of monoclonal antibodies produced by the hybridoma cells is determined by any of a variety of in vitro binding assays, depending on the nature of the antigen. For example, when a defined or purified antigen is available a radioimmunoassay (RIA) or enzyme-linked immunoabsorbent assay (ELISA) can be used.

By contrast, when an antigen is not well defined or is present in a heterogeneous mixture of potential antigens, as for example might be encountered with intact cells or cell extracts or fractions, the binding specificity is commonly determined by immunocytochemical or immunohistochemical staining using flow cytometry or microscope imaging analysis. This can be a multi-stage screening process, involving an initial semi-specific test for antibody production (i.e., positivity), followed by broader analysis of specificity.

Further determination of the specificity of each monoclonal antibody to a cell-associated antigen typically involves a comparative analysis of staining of a few to several dozen or more different cell types or tissue specimens in order to discern a pattern or range of antigen distribution. Thus, the total number of individual data determinations can be a substantial multiple of the original number of positive hybridoma wells or clones. The complexity of this process can pose a burden to the economics of labor, time and costs of screening efforts. The use of automation, robotics and imaging have made the latter process somewhat less tedious. However, the logistics of generating and interpreting large numbers of data determinations, whether positive or negative, represent significant rate-limiting steps in high-throughput screening efforts.

A number of attempts have been made to reduce the complexity and laboriousness of this screening and selection process by directly selecting hybridomas on antigen substrates based on the specificity of their cell surface antigen receptors. For example, Najbauer et al. (1986) Hybridoma 5:361, Najbauer et al. (1986) J. Neurosci. Research 15:415, Ossendorp et al. (1989) J. Immunol. Methods 120: 191, and Horton et al. (1989) J. Immunol. Methods 124:225, demonstrated enrichment of hybridoma cell lines (i.e., post-HAT selection and screening) specific for known antigens by binding to antigen-coated microtiter wells or magnetic beads. A major limitation of this type of approach was the necessity to use known, homogeneously purified, soluble protein antigens for coating the wells or beads.

Others have attempted to extend this direct selection of hybridoma cells to antigen-specific adherence on a cellular substrate. For example, Barral et al. (1997) J. Immunol Methods 203:103 demonstrated adherence to a tumor cell line of hybridoma cells derived from mice immunized against a heterogeneous protein extract of tumor tissue. A major limitation of this approach was the necessity to perform the adherence selections in microtiter plates in order to collect and further screen the individual secreted antibody products in each microtiter well for specificity by immunohistochemistry and ELISA. Another limitation of this approach was the very low efficiency of identifying antibodies of clonal origin. Only three specific hybridoma clones were confirmed by immunohistochemistry, despite the presence of antibodies reactive with the crude protein extract by ELISA in most of the wells. It can be inferred that this corresponded to an efficiency of clone identification of somewhere between 0.3 and 3% of the ELISA positive wells, depending on the total number of plates set up.

Direct selection of hybridomas based on specificity of cell surface antigen receptors has numerous inherent limitations. One limitation is that such antigen receptors represent the membrane-bound structural form of antibody whose presence and density have little or no correlation with the quantity of antibody that is secreted by the cell (which is not retained on the cell surface). In fact, the great majority of cells at the hybridoma/plasmacytoma level of maturation are known not to express detectable levels of surface antigen receptors by sensitive flow cytometric methods. Among those cells that do express detectable receptors, the receptor density on a substantial proportion can be too low to resist fluid shear forces and permit stable cell adhesion.

Another limitation of direct selection of hybridomas based on specificity of cell surface antigen receptors is that the steric accessibility of receptor antibody binding sites to antigenic determinants on a cellular substrate is highly constrained within a narrow defined distance from the hybridoma cell surface. Receptor antibody molecules are inserted in the membrane lipid bilayer at a uniform depth. This permits rotational and horizontal translational motion only in the plane of the membrane, and for vertical translational motion of antigen binding sites within the range of a parallel plane that is defined by the size of the extracellular heavy chain and the flexibility of the antibody hinge region (i.e., no more than 20-25 nm from the membrane surface). This can especially restrict cell-cell selection adherence approaches in which membrane antigenic determinants can be variably juxtaposed with other molecules of varying sizes, densities, combinations and conformations, and which can further be distributed in irregular membrane processes, blebs, microvilli, ruffles, etc.

To overcome some of the limitations of surface receptor antibody abundance or density for direct selection applications, several attempts have been made to retain the secreted antibody in physical association with their respective hybridoma cells. For example, Gray et al. (1995) J. Immunol. Methods 182:155 encapsulated the individual cells in microdroplets of agarose derivatized with a known antigenic peptide or with anti-mouse immunoglobulin antibody. The cells were allowed to secrete antibody, and antibody bound to the agarose was then labeled with a fluorescent complementary reporter reagent, either antigen or antibody, to permit sorting by flow cytometry. This method was shown to have utility for separating high antibody secretors away from poor secretors in a pre-established and cloned hybridoma line.

The work of Gray et al. (1995; ibid.) did not disclose whether such an approach could be used to screen for specific antibody producers involving undefined or heterogeneous antigen mixtures, or for cell-cell adhesion approaches. In fact, the increased diameter and mass of the encapsulated cell, which would further reduce steric accessibility and adhesion strength to antigens on irregular surfaces such as cells, pose limitations for this type of approach. Another limitation is that conditions to statistically prevent encapsulation of more than one cell per microdroplet require the vast majority of the capsules to be made empty, which in turn can greatly reduces the efficiency of the selection or sorting process.

PCT WO9409117 described a method for coupling the surface of various types of secreting cells with a moiety that is capable of capturing the product that is secreted and released from the cells. The product could then be used directly as a label, or alternatively, the product could be further labeled with labeling materials such as fluorophores, radioisotopes, chromophores or magnetic particles. The labeled cells could then be separated or detected using standard cell sorting techniques based on these labels, such as flow cytometry, magnetic separation, centrifugation, and the like.

However, PCT WO9409117 did not describe methods for carrying out cell selections without involving the use of a label moiety to label the captured product. Also, there is no disclosure of how such an approach could be used to screen and select individual specific antibody producers from among a mixed population of hybridoma cells in which the individual specificities are heterogeneous and undefined or unknown. This is a situation that commonly occurs with immunogens or antigens that are comprised of heterogeneous mixtures of molecules, such as with intact cells, cell extracts or fractions. In addition, there is no disclosure of an approach that could be used to screen and select specific antibody producers using unpurified or heterogeneous mixtures of potential antigens, such as with intact cells or cell extracts or fractions. Further, no distinction is made between the disclosed, relatively simple process of capturing antibody for the purpose of labeling with a substance in solution or suspension, versus the undisclosed, more complicated process of capturing an antibody with effective steric spacing and accessibility for the purpose of enabling hybridoma selections by cell-cell adhesion. The means to achieve the latter end would not be within the skills of those in the art without further conceptual insight and experimentation.

Whether the direct antigen-specific selection of hybridoma cells involves a surface receptor antibody or a captured secreted antibody approach, the current art fails to anticipate numerous complicating variables that can greatly reduce the efficiency of cell selection on heterogeneous antigen substrates, such as those comprising intact cells or cell extracts or fractions. For example, heterotypic cell-cell interactions mediated through adhesion molecules such as integrins and other ligand-receptor pairs are frequently observed in vitro between cells of disparate lineages. Mature B lymphocytes typically interact with T lymphocytes, macrophages, and dendritic cells through adhesion molecules when initiating immune responses. B lymphocytes (from which hybridomas are derived) at various states of maturation and activation also form adhesive interactions with cells of endothelial and fibroblastic origin and presumably many other cell types. Thus, without the inclusion of new methods to prevent such heterotypic interactions between hybridoma cells and cell-derived heterogeneous antigen substrates, only a tiny minority of adhesive interactions can occur through antigen-specific mechanisms.

An alternative approach to the traditional generation of monoclonal antibodies from hybridomas has been described in which the immunoglobulin variable region cDNAs obtained from single, isolated cells producing antibodies of defined specificities are molecularly cloned and expressed (for example, Lagerkvist et al. (1995) BioTechniques 18:862 and Babcook et al. (1996) Proc. Natl. Acad. Sci. USA 93:7843). The single lymphocytes in the methods exemplified in Babcook et al. (1996) ibid., were antibody-forming cells (i.e., AFC, also known as plaque-forming cells or PFC) that produced zones of lysis of erythrocytes coated with defined antigens in the presence of complement, using a standard hemolytic plaque assay. The AFC were then isolated from the centers of these zones by micromanipulation. In principle, such methods could be applied to the extraction of either mRNAs or genomic DNAs encoding the specific antibody genes from AFC or hybridoma cells, and also to the screening against nucleated cell targets.

Although molecular cloning and expression methods can be useful with AFC screened against a homogeneous or defined antigen preparation by hemolytic plaque formation, they would be highly inefficient with AFC screened against heterogeneous mixtures of antigens that are individually unknown or undefined. Traditional plaque assays and similar methods known in the art lack the capability to distinguish any one specificity from among a much larger multitude of other reactive specificities. In addition, a single AFC can be tested only one time. Thus, obtaining sufficient amounts of antibody for further screening would necessitate the molecular cloning and expression of antibodies from every AFC in order to analyze the entire repertoire of specficities from an immunized host. In the cited example of Babcook et al. (1996) ibid., the number of AFC to defined antigens or peptides exceeded the number of specific hybridoma clones obtained from mice by a factor of 10 or greater, numbering in the tens of thousands of AFC. The majority of these AFC are likely to be identical cell progeny of a smaller number of antigen-specific B cell clones, and are thus redundant. Thus, analyzing the full repertoire of AFC specificities of even a single immunized animal in this way, most of which are redundant or irrelevant, can be an impractical if not an impossible task.

In light of the limitations of the above-cited methods, it should be evident that satisfactory methods are not available for screening and selection of antibody-secreting cells to undefined antigens contained within heterogeneous antigen mixtures on substrates, including intact cells, cell extracts or fractions. Further, no pre-existing methods address the issue of steric accessibility of either surface antigen receptor or captured secreted antibody for selections by cell-cell adhesion. Establishing new methods for such perceived deficiencies would require concepts and methods beyond the current art.

### SUMMARY OF THE INVENTION

Embodiments of this invention include compositions, methods and kits suitable for detecting desired antigens that are present within a mixture of both desirable antigens and undesirable antigens, and for screening and selecting cells that are producing antibodies directed toward those antigens.

In one aspect of the invention, embodiments include libraries of polyclonal antibodies (“polyclonal antibody libraries” or “PALS”) are prepared against heterogeneous mixtures of antigens. The antigens can be derived from intact cells, cell extracts, cellular organelles, cellular molecules, cellular fractions, cellular digests, or other cellular components. Cells can be either naturally occurring, or can be transgenic or recombinant. For example, cells can be made from subtracted DNA libraries that have a subset of normal cellular constitutents. A heterogeneous mixture of antigens derived from a cell type not having a desired antigen can be used as an immunogen. Lymphocytes isolated from animals having such an immunization can be fused with myeloma cells, or can be immortalized using other methods to provide a mixed pool of renewable antibody producing cells. This mixed population of cells can be expanded and grown to produce a large, mixed population of antibody producing cells, each of which produces antibodies directed against a certain set of antigens, including previously unknown and/or undetected antigens. Antibodies produced by such populations of cells can be collected into a PAL. The libraries so produced can be designed not to contain an antibody directed against an antigen of interest.

A PAL can then used as a mask to block those antigenic sites on a target that are recognized by antibodies within the pool. For example, a target can be a specific tumor cell. If PAL is prepared against antigens from normal or non-tumorous cells, then the components of the PAL can bind to and “mask” those antigens if they are present in the tumor cell. Thus, non-normal or unique tumor antigens will be unoccupied in the heterogeneous antigen substrate, and a test antibody, if it binds to the substrate, is more likely to recognize a non-normal antigen, thereby decreasing the probability of “false positives” in a screening assay for antibodies directed toward such unique antigens. Such masked antigen substrates then can provide a means for evaluating antibody-producing cells that can serve as a source of monoclonal antibodies, for example, for their ability to recognize antigens not present in normal cells. Any antibodies from the antibody-producing cells that are directed toward antigens present on normal cells will therefore not be bound, because antibodies from the PAL will have already substantially bound to those antigens and thereby block subsequent binding of a test antibody. Therefore, any binding of test antibodies to a PAL pre-treated antigen substrate will reflect the binding to a non-normal antigen.

In another aspect of the invention, these test antibodies can be secreted from antibody-producing cells and can be captured in physical association with the surfaces of the cells of origin. Antibody-producing cells can be prepared against heterogeneous mixtures of antigens. A flexible molecular scaffold can be constructed on the surface of each cell that provides to the captured secreted antibody a high degree of rotational and horizontal translational motion over the plane of the secreting cell surface membrane, as well as providing a high degree of vertical translational motion throughout a parallel plane extending over a large effective binding distance from the cell surface. This construction can provide a high degree of steric or spatial accessibility of the captured secreted antibody to any potential specific antigen in a heterogeneous antigen substrate.

These antibody-producing cells with their captured secreted antibodies can interact directly with the antigens on a heterogeneous antigen substrate toward which they are directed, and can adhere in an antigen-specific way. The cells adhering to antigens on the substrate can then be physically separated from the nonadherent cells to obtain antigen-specific selections. By way of example only, such separation techniques used in the art include cell panning, cell affinity chromatography, magnetic cell sorting, fluorescence activated cell sorting, and the like. The specifically adherent modified secreting cells can then be enumerated, collected or harvested at some desired timepoint, or can be cultured in situ for some desired period of time.

In a further aspect of the invention, embodiments of antibody-producing cells with their captured secreted antibodies (“modified antibody-secreting cells”) can interact with a PAL pre-treated antigen substrate to obtain a more narrow or restricted range of interaction that is limited to antigens that are not masked by antibodies of the PAL. Antibodies can then be obtained with specificities that reflect that of the adherent modified antibody-secreting cells following the collection or harvesting of these cells or their progeny. Collecting these adherent modified antibody-secreting cells individually can be used to obtain monoclonal antibodies. When these cells have replicative potential, such as with hybridoma cells, further cell cloning or subcloning can be done by in vitro culture followed by expansion in vitro or as ascites in vivo. Alternatively, these adherent modified antibody-secreting cells can be collected and grown as pools of polyclonal cells that can be used to obtain another PAL. The secreted antibody product can then be collected from the culture medium or ascites, concentrated or purified, and further chemically or enzymatically modified as desired. Alternatively, the nucleic acids encoding the secreted antibody product can be purified and subjected to genetic cloning, splicing or recombination, transfection or expression in other cell systems as desired.

Kits comprising a PAL, PAL producing cells, heterogeneous antigen substrates derived from desired targets, and methods of using those compositions and kits can allow desired monoclonal antibodies directed at novel antigens to be discovered with substantially greater efficiency.

Thus, in various embodiments, the invention encompasses methods for preparing heterogeneous antigen mixtures onto solid substrates for the purpose of reacting those antigen mixtures with a PAL; for tailoring the specificities of a PAL so that the antibodies can bind to a majority of different antigens on a substrate except for those antigens having a desired property (e.g., are tumor-cell specific); for using the PALs to bind or mask various antigens on a substrate; and for preparing PALs so that they lack an antigenic or structural feature that can be present on a captured secreted antibody. Such structural or antigenic differences can permit the captured secreted antibody to be distinguished from the PAL antibodies bound to the substrate.

Yet further aspects include embodiments of methods for maintaining, replenishing, and/or expanding PALs. Still other aspects include compositions of matter comprising PALs and/or mixed pools of monoclonal cells that produce a PAL, including uncloned or cloned, and immortalized cell lines, including hybridomas, transfectomas or bacteriophage that are sources of PALs.

Additional aspects include embodiments of PALs having specificities tailored for antigens that are structurally distinguishable from antigens against which test antibodies are prepared.

In still further aspects, this invention includes embodiments of methods to facilitate the screening and production of monoclonal or polyclonal antibodies directed toward specific, desired antigens that can be present in homogeneous or heterogeneous form or in mixtures of antigens from specific target cells, cell populations, cellular organelles, digests, tissues, organs or organisms.

The compositions and methods of this invention can be used to reduce the complexity and time necessary for generating, screening and selecting monoclonal antibodies and antibody-producing cells compared to conventional methods. One application of the compositions and methods of this invention is in high throughput screening of antibodies for antigen discovery and/or therapy using monoclonal or polyclonal antibodies as therapeutic agents or to target therapeutic agents to specifically desired cell types.

### BRIEF DESCRIPTION OF THE DRAWINGS

FIG. 1 depicts a scheme for a “flexible antigen-binding scaffold” (FLABS) assembly on the surface of a hybridoma cell secreting mouse IgG antibodies, showing the capture of the secreted antibody in association with the cell surface.

FIGS. 2a-2d depicts schematically various embodiments of this invention having a substrate with heterogeneous antigens expressed on a cellular lawn thereon. Antibodies in a “polyclonal antibody library” (PAL) interact with their respective antigens in the substrate. Modified antibody-secreting cells then interact with the PAL-treated substrate and can adhere or not adhere depending on whether their antigens are not masked or masked, respectively, by the PAL.

FIG. 3 depicts a schematic example of the range of overlap in antigen recognition that can occur between antibodies in a PAL compared to the totality of antibody specificities in a panel of modified antibody-secreting cells being screened, showing specificities that are overlapping and specificities that are unique to each group.

FIGS. 4a and 4b depicts schematically the interaction with and masking of antigens present in a heterogeneous antigen substrate by antibodies in a PAL, leaving not masked another antigen which remains accessible for binding by a modified antibody-secreting cell. The bound antibodies of the PAL are either not covalently attached to their respective antigens, or they are covalently attached with chemical cross-linkers.

FIG. 5 is a schematic representation of a overall process for the selection of FLABS-modified antibody-secreting cells on a PAL-modified antigen substrate.

### DETAILED DESCRIPTION

The invention encompasses methods for making modifications to both the antibody-secreting cells and to the antigen substrate and then using these modified materials in combination so as to obtain a new composition that produces a synergistic result. In brief, an object of the secreting cell modifications is to capture secreted antibody in monoclonal form in physical association with its cell of origin, and thereby enable antigen-specific cell adhesion and selection through this antibody. Also briefly, an object of the antigen substrate modifications is to block or mask a substantial portion of the potentially reactive antigens using polyclonal mixtures of antibodies or antibody libraries that contain common or recurring specificities. A result of combining these two entities is to achieve the adherence of the modified secreting cells to the modified antigen substrate that is selective for the remaining unblocked antigens that represent novel or unique specificities. Recovery of these selectively binding adherent cells for various subsequent antibody production processes will thereby be made more efficient.

Several definitions apply to the methods described herein as follows:

The term “antibody” is used here in the broadest sense and specifically includes polyclonal antibodies, monoclonal antibodies (including full-length monoclonal antibodies), multispecific antibodies(e.g., bispecific antibodies), and single domain antibodies, phage antibodies and antibody fragments. The term “antibody fragments” means a portion of a full-length antibody, generally the antigen binding or variable region thereof, and that lacks all or part of the Fc region. Examples of antibody fragments include Fab, Fab′, F(ab′)₂, Fv fragments, single-chain antibody molecules and single domain antibody molecules.

The terms “specificity” and “specificities” include the degree to which a given antibody or mixture of antibodies react with certain antigens. Thus, mixtures of antibodies with overlapping specificities can react with a number of different antigens in common, yet one mixture still can react with antigens not recognized by other mixtures of antibodies.

The term “antigen” as used herein includes a molecule or portion of a molecule (epitope) that can interact with and bind to a recognition site on an antibody. The term includes materials that react strongly and with high specificity, and also includes materials that react weakly and/or with low affinity to an antibody. The term “antigen” also means a molecule or a portion of a molecule (epitope) that can, either by itself or in conjunction with an adjuvant or carrier, elicit an immune response (also called an “immunogen”).

The terms “heterogeneous antigen” and “heterogeneous antigen mixture” includes a mixture of two or more antigens used for immunization or for detection (e.g., screening).

The term “heterogeneous antigen substrate” includes a mixture of two or more antigens derived from a source and are applied to a detection surface, such as, by way of example, a slide, wafer, dish, and the like.

Included in the term “intact cells” as used herein include eucaryotic or procaryotic cells that can naturally express, or can be genetically modified to express, or can be coupled with, any antigen or antigens of interest. The term “cell extracts or fractions” broadly includes cell-derived organelles, proteins, glycoproteins, glycolipids, nucleic acids and other cellular components or antigens. Any of these can be extracted using aqueous, detergent or organic solvents, freeze-thawing, sonication, cavitation, secretion, shedding, enzymatic digesion or other methods, and can be fractionated by various methods such as by size, solubility, density, charge, affinity or chemical labeling, antibody or lectin binding, chemical or enzymatic fragmentation, differential display or expression, molecular cloning efficiency or mutagenesis, etc. Any of these above materials can be a source of immunogens or antigens.

A. Modifications to the Antibody-Secreting Cells

In some embodiments, the capturing of secreted antibody in physical association with its secreting cell of origin employs a method for attaching a capturing moiety to the cell surface. This secreting cell can be an immortalized hybridoma cell, an antibody-forming cell (AFC) or plaque-forming cell (PFC), or some other cell type that is be engineered to secrete antibodies, such as a cell transfected with nucleic acids encoding antibodies or antibody specificities (e.g., transfectomas). For most purposes, and especially when the specificity of the secreted antibody can not yet be defined, this capturing moiety is generally an antibody (i.e., a “capturing antibody”) specific for some antigenic feature of the secreted antibody. Examples of common antigenic features on the secreted antibody include immunoglobulin class, subclass or isotype, or allotype markers of the animal species or strain. The term “antibody” as used herein is intended to include monoclonal and polyclonal antibodies, chimeric antibodies, antibody fragments, multispecific antibodies (e.g., bispecific antibodies) and polymeric forms of antibodies.

In one embodiment, the said antigenic feature is located in a region of the secreted antibody molecule that is separate from its antigen binding region (i.e., the Fab arms), so as to help orient the antibody in a way that allows relatively unhindered flexibility of its hinge region (in immunoglobulin classes that have hinge regions). Preferably also, the antigenic feature can serve to distinguish secreted antibody from surface receptor antibody. Non-exclusionary examples of commercially available reagents that can serve this purpose include Fc-specific anti-immunoglobulin class antibodies, such as anti-mouse IgG-Fc or anti-human IgG-Fc for secreted mouse or human antibodies, respectively.

An alternative to the said antigenic feature is a structural feature that preserves many of the desirable aspects of broad immunoglobulin binding reactivity and of binding orientation. Useful examples of alternatives to antibody for this purpose include staphylococcal protein A or protein G, which have specificity for the Fe region of IgG molecules of most mammalian species.

A capturing antibody can be attached to the secreting cell through an anchoring moiety on the cell surface. This anchoring moiety can be created in several different versions. In one version, this can be any defined cell surface antigen which is expressed on differentiated B lymphocytes, including hybridoma cells or plasma cells, such as major histocompatibility or MIIC antigens, pan-leucocyte marker CD45, or other “cluster of differentiation” (“CD”) antigens. Ideally, the antigen is present in some relative abundance and an antibody specific for the antigen is also available to form part of the means for connecting to the capturing antibody.

In another embodiment, the anchoring moiety can be a common biochemical feature of a group of cell surface molecules. For example, cell surface glycoproteins or glycolipids can share carbohydrate linkages that are recognized by a specific lectin, and the lectin can be used as part of the means for connecting to the capturing antibody. A variety of lectins suitable for this purpose, along with descriptions of their specificities and molecular properties, are available from various commercial suppliers (e.g., Sigma Chemical Co.).

In still another embodiment, the anchoring moiety can be introduced through ionic interactions involving negatively charged sugars such as sialic acids on the cell surface. Various polycations are known to interact with these charged sugars, including polylysine, protamine sulfate, and chitosan. These polycations can serve as a backbone or framework to which additional chemical modifications can be made in order to introduce a member of a specific binding pair that forms part of the means of connecting to the capturing antibody.

In yet another embodiment, the anchoring moiety can be introduced through hydrophobic interactions with the cell membrane lipid bilayer. For example, synthetic molecules are commercially available that contain both a hydrophobic component such as a fatty acid tail for insertion into the membrane, and also a member of a specific binding pair that can form part of the means of connecting to the capturing antibody.

In any of these embodiments, the member of the binding pair can comprise a hapten which is bound by specific antihapten antibody, a specific or flag peptide recognizable by complementary antibody, biotin which is bound by avidin or streptavidin, NTA which binds to polyhistidine peptides, or a sugar sequence or linkage which binds to a complementary specific lectin. The complementary member of the binding pair can then serve to form part of the means of connecting to the capturing antibody.

In another embodiment of the invention, an anchoring moiety is introduced through chemical derivatization at the cell surface. Through such means, any of a variety of compounds such as haptens, biotin, or peptides can be coupled so as to comprise a member of a specific binding pair. Especially preferred among the haptens are fluorescein, biotin, trinitrophenol, and digoxigenin, and any of their structurally related analogues or derivatives. Chemical coupling is advantageous in that it allows the use of uniform coupling conditions for any cell type or experiment, and also allows the number or density of such molecules to be adjusted to a higher or lower degree as appropriate for optimizing antibody capture. Quantitative measurement of the level of derivatization can be performed by an assay such as flow cytometric staining using antibody, avidin, or other complementary member of the binding pair where appropriate.

Methods for chemical coupling for the purpose of introducing a member of a specific binding pair are known in the art and include the use of compounds containing reactive succinimidyl esters, imidoesters, or aldehydes for coupling to protein amino groups; malemidyl esters, haloacetates, or pyridyldithio groups for coupling to protein thiols; hydrazides for coupling to oxidized sugar alcohols on carbohydrates; carbodiimides for coupling to protein amino or carboxyls; and aryl nitrene or carbene photaffinity groups for insertion into C—C or C—H bonds. Ideally, the coupling procedures are carried out under physiological conditions that do not reduce cell viability and functional activity. Suitable procedures can be readily adapted by reference to Hermanson in: Bioconjugate Techniques (1996; Academic Press), or otherwise devised by those skilled in the art. Suitable chemical analogues and derivatives are available from a number of commercial sources, including Molecular Probes and Pierce Chemicals.

Means of connecting the capturing antibody or moiety to the anchoring moiety involves the addition of a bridging or linking moiety. The bridging moiety is a critical component of the configuration for ultimately providing access of captured secreted antibody to antigens that can be present in substrates comprised of heterogeneous mixtures of other potential antigens, such as on intact cells or in cell extracts or fractions. Bridging moieties are chosen with consideration to the following: (a) multiplying the number of capture antibodies and thus the potential for capturing secreted antibody product, (b) providing spatial separation between the secreting cell surface and the capturing antibody and thus the secreted antibody product, (c) maintaining the captured secreted antibody product in effective steric orientation for access to any potential antigen in the antigen substrate, (d) providing the captured secreted antibody product with high freedom of mobility or flexibility in horizontal and vertical dimensions for access to antigen, and (e) avoiding or minimizing intermolecular crosslinking of neighboring anchoring moieties on the secreting cell, which could lead to cytotoxic effects or to modulation (i.e., stripping or loss) of antibodies from the surface.

A variety of substances can perform these bridging functions to varying degrees. For example, branched polymers, including modified dextran molecules, polyethylene glycol, polypropylene glycol, polyvinyl alcohol, and polyvinylpyrollidone, can be effective in multiplying antibody capture potential and extending the captured secreted antibody away from the secreting cell surface. Similar functional attributes can apply to substances having a more solid chemical composition or a more spheroidal shape, including particles, beads or dendrimers of varying sizes, especially those ranging from 10 to 500 nm average diameter. Any of these substances can be further derivatized according to known methods in order to attach a member of a specific binding pair. However, the structural rigidity of these substances in solution can limit somewhat the freedom of antibody mobility for access to antigens on intact cells, or for minimizing crosslinking of anchoring moieties on the cells.

Antibodies may be desirable because they fulfill many of functions of a bridging moiety. Their size and molecular flexibility in the hinge region (among those immunoglobulin classes that have hinge regions) and on through their Fab arms can impart a high degree of horizontal and vertical translational mobility to the capturing antibody and thus to the captured secreted antibody. In addition, their multivalency can multiply the potential number of captured secreted antibody molecules. Further, conditions can be devised by those skilled in the art to minimize the potential for crosslinking anchoring moieties by taking advantage of their solution solubility at high concentration and their fast diffusion kinetics (relative to larger particles).

Antibodies can be used as bridging moieties in any of several different chosen modes. For example, they can be used either directly as monomeric entities or as multimeric antibody chains or polymers made up of the same or different antibodies. In one series of embodiments, the antibodies are polymeric so as to further extend the spacing between the cell surface and captured secreted antibody product, further multiply capturing and secreted antibody binding potential, and further increase the overall flexibility of the antigen binding scaffold comprised of the anchoring, bridging, and capturing moieties along with the captured secreted antibody. Polymeric antibodies can be obtained as naturally secreted IgA or IgM molecules having a single antigen specificity, or they can be made artificially as bispecific or bifunctional antibodies. Further, antibodies can be incorporated into a bridging composition that also includes other macromolecules, polymers, beads, particles, or dendrimers to further increase the access of captured secreted antibodies to potential antigens.

Other multivalent binding proteins in monomeric or polymeric form, such as avidin or streptavidin and recombinant modified variants thereof, can also serve as useful bridging moieties in place of or in addition to antibodies.

For the purpose of obtaining antigen-specific adhesion, a captured secreted antibody product by the cell of origin should be substantially homogeneous or monoclonal, even when that cell is present among a polyclonal mixture of cells secreting heterogeneous other antibody products. Practicality of purpose takes into consideration that antigen-specific adhesion of the cells to an antigen substrate can be typically multivalent, and so can require significant or substantial homogeneity of capture rather than absolute homogeneity. Intrinsic properties of the cells that favor homogeneity include the high rate of antibody secretion, (normally in the range of hundreds to thousands of molecules per second for hybridoma and plasma cells), along with local high concentration and mass action effects. Limiting the incubation conditions to a period of time that is sufficient but not much in excess for what is needed for the capturing antibodies to bind secreted antibody further favors this result. Generally the incubation time for antibody-secreting cells ranges between 5 minutes and 2 hours. However, other time periods can be used. The optimum time can be determined for a given antibody secreting cell population and capturing configuration by using known techniques such as fluorescent staining of the bound secreted antibody and flow cytometric analysis to measure antibody secretion and capture kinetics.

To achieve efficient antibody capture on any given secreting cell with less potential risk of cross-contamination by antibodies originating from other secreting cells that can be nearby, additional manipulations can be made. This can include addition to the incubation medium of a substance that slows diffusion of the secreted product from the cell of origin or that holds the cells in suspension apart from neighboring cells. Substances that inhibit diffusion or suspend cells in liquid medium and are non-toxic to cells are known in the art. These include, for example, a variety of substances that partially or incompletely gel, such as low melting agarose, gelatin, methylcellulose, or alginate, or that have adjustable densities, such as percoll, ficoll, albumin, or sucrose. By adjusting the viscosity, permeability or density of the medium, the local capture by an antibody-secreting cell can be optimized. Preferably, after the incubation the gel is solubilized or the density medium diluted to allow for the dispersion and recovery of the cells in preparation for further manipulations. The means to disperse and recover the cells are within the skills of those in the art.

It will also be apparent to those skilled in the art that various changes or substitutions can be made in the foregoing descriptions that do not substantively alter the final outcome. These include, but are not limited to, specific members of the binding pairs, chemical analogues or derivatives of either binding pair member, incubation or reaction conditions (including time, temperature, pH, concentration, etc.), and order of addition of any of the moieties, including adding these as pre-formed or assembled complexes. When pre-formed complexes are used, they can be in the form of soluble immune complexes or can be coupled to particles or beads with colloidal properties (i.e., 500 nm or smaller) or suspensions up to 1 to 2 um.

In addition, with further modifications according to the aforementioned methods, certain of the compositions and methods of PCT WO9409117 can also be found suitable for the present purposes and this is incorporated herein by reference.

For convenience of reference, such a fully assembled configuration of antibodies is herein termed a “flexible antigen binding scaffold” or “FLABS.” This term is meant to be used in the broadest sense consistent with its composition and use, and not to be construed as limiting its use for any specific application. It can be possible to conjure other terms that would be equally descriptive of the same compositions and methods.

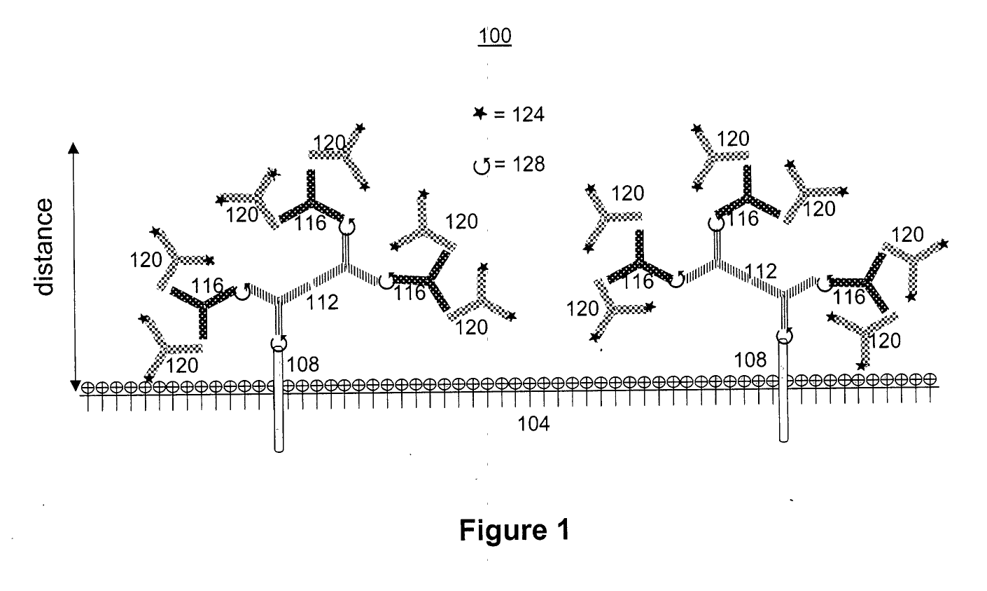

FIG. 1 depicts schematically a nonlimiting embodiment of this invention 100 with a FLABS assembly configuration on the surface of a hybridoma cell secreting mouse IgG antibodies, wherein the cell surface membrane 104 is associated with an anchoring moiety 108 of fluorescein hapten conjugated to cell surface proteins, a bridging moiety 112 of polymeric mouse IgA anti-hapten antibody, a capturing moiety 116 of fluorescein hapten-conjugated sheep anti-mouse IgG-Fc antibody, captured secreted antibody 120, and antigen binding sites of the captured secreted antibody 124. Hapten-anti-hapten linkages 128 are also indicated. Note that each antibody molecule in the FLABS may have a flexible hinge region and that the number of antigen binding sites of the captured antibodies can be a multiple of the number of anchoring moieties.

### EXAMPLES

Three illustrative and nonlimiting examples of methods for the capture of secreted antibody products on hybridoma cells and antibody-forming cells (AFC) for the purpose of antigen-specific cell adhesion and selection are as follows:

### Example 1

### Modification of Hybridoma Cells for Secreted Antibody Capture

Hybridoma cells were cultured in bulk in HAT selection medium for several days after fusion of a drug sensitive mouse myeloma cell line with lymphoid cells from a mouse immunized with human prostate tumor cells. After collection of the viable hybridoma cells and washing them free of debris and spent medium by centrifugation, they were then reacted with a succinimidyl ester of biotin for approximately 30 minutes in an inert buffer of neutral or slightly alkaline pH to derivatize protein amino groups and generate the anchoring moiety. Following additional washing of the cells to remove unconjugated biotin, they were then reacted for approximately 30 minutes with streptavidin to generate the bridging moiety. Following further washing of the cells to remove excess free streptavidin, the capturing moiety was generated by reacting with a biotin-conjugated anti-mouse IgG-Fc antibody. The cells were then incubated at physiological temperature (usually 37° C.) to allow antibody secretion and capture. At the end of the secretion phase the cells were then chilled to prevent further secretion. A secretion-blocking agent such as brefeldin A was optionally added to allow more convenient manipulation of the cells at warmer temperatures.

### Example 2

### Alternate Modification of Hybridoma Cells for Secreted Antibody Capture

Hybridoma cells were obtained and manipulated in a manner similar to examplee 1, but the anchoring moiety consisted of protein amino groups derivatized with a succinimidy ester of fluorescein, the bridging moiety was a polymeric mouse IgA anti-fluorescein antibody, and the capturing moiety was a fluorescein conjugated anti-mouse IgG-Fc antibody, respectively.

### Example 3

### Modification of Normal AFC for Capture of Secreted Antibody

Spleen and lymph node cells were harvested from mice 5 days after the last of a series of immunizations. A fraction of large-sized cells substantially enriched in differentiated B cells or plasmacytic cells (i.e., AFC) was obtained by velocity sedimentation through a low density medium at unit gravity, or through a density gradient at low centrifugal force (In: Mishell and Shiigi, Selected Methods in Cellular Immunology (1980), W.H. Freeman and Company, pp. 186-96). Alternatively, the enriched fraction was obtained by flow cytometry sorting and gating on cells with high forward light scatter. The cells were then treated in the same manner as in the second example to generate the anchoring, bridging and capturing moieties.

B. Modifications to the Antigen Substrate.

The methods provide for the production of renewable libraries comprised of soluble polyclonal or oligoclonal antibody mixtures with multiple specificities directed toward a heterogeneous subset of antigens present in or on intact cells, cell extracts, cell fractions, cellular organelles, and/or digests. For convenience of reference, such an antibody library is herein termed a “PAL.” This term is meant to be used in the broadest sense consistent with its composition and use, and should not be construed as limiting its use for any specific application. Other terms can be defined that are equally descriptive of the same compositions.

A PAL can be reacted with intact cells, cell extracts or fractions, tissue sections or the like that are attached to a surface or matrix by suitable means to produce an antigen substrate. The surface or matrix can be any material known in the art to adhere to antigens sufficiently to remain adhered even after being subjected to conditions of washing, with solutions necessary for analysis by antigen blocking and subtraction methods. By way of example only, such substrates can be in the form of dishes, multiwell plates, films, membranes, ribbons, beads, particles, capillary tubes, etc., and can be chosen to be impermeable or porous to liquids. The techniques used for attachment are chosen for compatibility with the chemical composition of the surface, and can include direct chemical derivatization, covalent crosslinking, indirect coupling such as by antigen-antibody mediated or biotin-avidin bridging, noncovalent coupling by ionic or electrostatic interactions, hydrophobic interactions, copolymerization, gel entrapment, drying, surface tension effects or other techniques known in the art. Suitable procedures for derivatization, for example, can be found by reference to Hermanson in: Bioconjugate Techniques (1996; Academic Press), incorporated herein fully by reference.

If the antigen substrate is a relatively flat surface, then the antigens can be distributed randomly or organized on a grid, so that each position on the grid can be determined or can be placed into registry with the screening monoclonal antibodies or other compounds that are brought into contact. An antigen substrate comprising a cellular “lawn” or monolayer is a convenient format for screening large numbers of candidates. Alternatively, particles and beads can be chosen from among those that impart useful density, magnetic or optical properties to the screening system.

In one embodiment, a PAL is used in conjunction with the screening or testing of antibody-secreting cells generated against immunogens comprising heterogeneous mixtures of potential antigens. It should be understood that heterogeneous mixtures of potential antigens can be obtained even when a given molecule is defined or cloned, if it is expressed, coupled with or otherwise present among a heterogeneous mixture of other molecules (e.g., intact cells) used for immunization. In preparation for such screening applications, the immunogen used to generate PALs and the immunogen used to generate antibody-secreting cells being tested can be chosen such that each can generate a range of antigen specificities that can overlap with the specificities of others.

Polyclonal antibody libraries can be used to “mask” antigens in heterogeneous antigen substrates. Components of a PAL can be brought into contact with and can bind to a plurality of different antigens present in the heterogeneous antigen substrate that is then used to screen the test antibody-secreting cells. Said antibody-secreting cells can be modified as described herein to capture their secreted antibody products monoclonally on the surfaces of the cells of origin. The subsequent binding of these modified antibody-secreting cells to a PAL-treated antigen substrate can be at least partially inhibited or blocked if the cells have same or overlapping specificity as those of the PAL. However, the PAL does not substantially interfere with the binding of other modified antibody-secreting cells directed to the desired antigens not present in the immunogenic antigen mixture used to produce the PAL. These desired antigens can include, for example, antigens with a more restricted expression pattern.

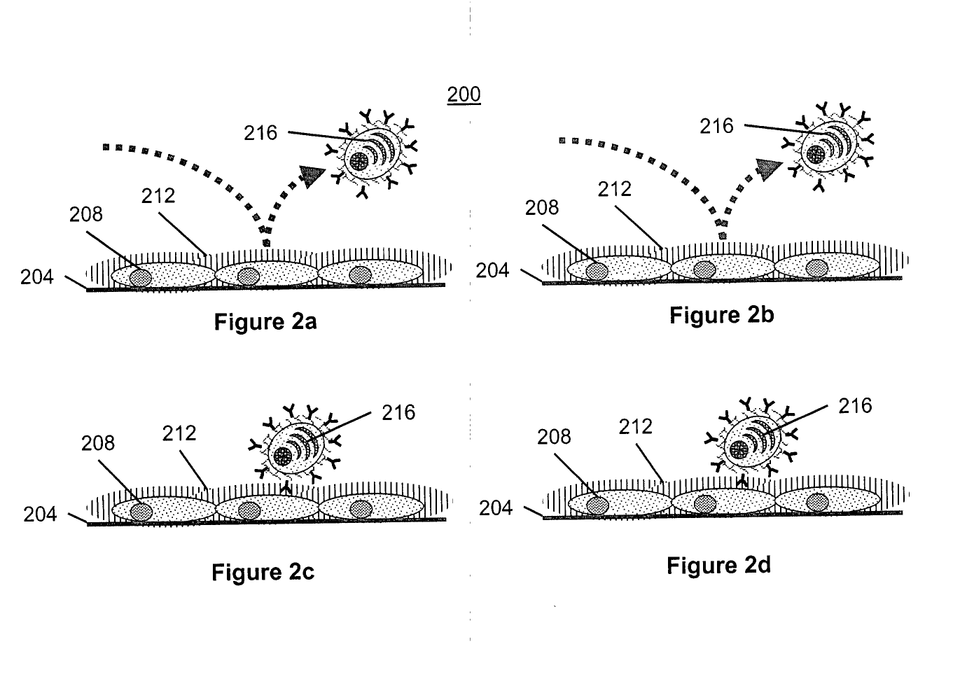

FIGS. 2a, 2b, 2c, and 2d depict schematically various embodiments of this invention 200 having a substrate 204 with heterogeneous antigens 208 expressed on a cellular lawn thereon. Antibodies in a PAL 212 interact with their respective antigens in the substrate and modified antibody-secreting cells 216 then interact with the PAL-treated substrate. In FIG. 2a, a modified antibody-secreting cell has specificity for an antigen that is masked by antibodies in the PAL and is unable to adhere to the substrate. In FIG. 2b, a modified antibody-secreting cell having specificity for an antigen is not present in the substrate is unable to adhere to the substrate. In FIG. 2c, a modified antibody-secreting cell having specificity for an antigen that is not masked by antibodies in the PAL and is able to adhere to the substrate. In FIG. 2d, a modified antibody-secreting cell having specificity for an epitope of an antigen that is not masked by antibodies in the PAL and is able to adhere to the substrate.

By applying suitable techniques to enumerate the adherent modified antibody-secreting cells, such as by direct visualization on the substrate or after collection from the substrate, it can be shown that PAL can diminish or, in some cases, abolish the contributions of antigens that are recognized by both the polyclonal antibodies and the modified antibody-secreting cells. This antigen subtraction effect has significant benefits for the screening and selection of monoclonal antibodies, hybridomas, AFC and other antibody-secreting cells.

One way of quantifying the amount of the subtraction effect is through the use of a “signal subtraction ratio” or “SSR.” A signal subtraction ratio is calculated by determining the number of modified antibody-secreting cells that adhere specifically to an antigen substrate which is not exposed to the PAL (“unmodified antigen substrate”), divided by the number of modified antibody-secreting cells which adhere to an antigen substrate (“modified antigen substrate”) that has antibodies of the PAL bound thereto. An SSR of 1 means that the same number of modified antibody-secreting cells react positively to the unmodified and modified antigen substrates, and that none of the antibodies in the PAL masks antigens recognized by the modified antibody-secreting cells. An SSR of greater than 1 means that at least some of the antibodies in the PAL decrease the binding of modified antibody-secreting cells to antigens.

The amount of signal subtraction can be quite substantial. Desired SSR can be in the range of greater than about 1 to over about 1000, alternatively from 1.5 to about 100 and yet alternatively, from about 2 to about 50. In yet further embodiments, the SSR can desirably be in the range of about 5 to about 20, and in still further embodiments, in the range of about 5 to about 15. It can be especially desirable if the SSR is above about 10, although any SSR greater than one represents an improvement in the screening efficiency. A high degree SSR can be especially desired with complex antigen mixtures such as intact cells.

This antigen subtraction effect using PALS can substitute for the complicated interpretations of immunocytochemical or immunohistochemical staining patterns that are commonly encountered when screening soluble monoclonal antibodies in the absence of PALs. In addition, a single positive binding result for a given modified antibody-secreting cell in the presence of PALs of this invention can replace the results of staining of dozens or more different tissue specimens with the secreted monoclonal antibody product of that same cell in the absence of PALS.

A novel feature of compositions having a PAL is that although the antibodies present have an overall range of reactivity, the specificities of each individual antibody in the PAL need not be known or defined. Unlike conventional mixtures of monoclonal antibodies, the antibody libraries of this invention need not be directed to pre-determined antigens. The antibody libraries are effective in part because the antibody pool can be biased towards common or recurring antigens. The antigens can exist in a wide range of cell types, as well as specificities that can have more restricted ranges of cell distribution.

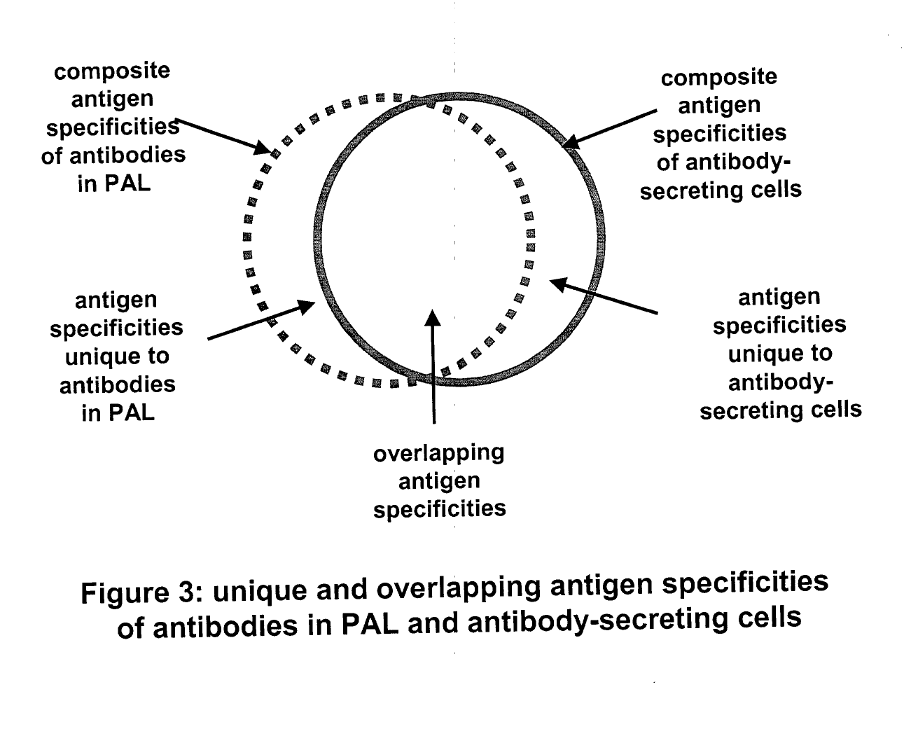

FIG. 3 depicts a schematic example of the range of overlap in antigen recognition that can occur between antibodies in a PAL compared to the totality of antibody specificities in a panel of modified antibody-secreting cells being screened. Thus, depending on the quality or quantity of the overlapping specificities, a skilled worker in the art can be able to control the level of stringency in the screening to desired effect (e.g., greater overlap can give higher stringency, and vice-versa).

It can be appreciated that a PAL of this invention can be easily renewed or replenished in the form of immortalized, permanent lines including hybridomas. This replenishment can be accomplished without In contrast, polyclonal antiserum derives from a single animal or group of animals each producing an individual spectrum of antibodies that may not be reproducible from one animal to the next, or even in the same animal over time. Collection of polyclonal antiserum is thus limited in volume and limited by the useful lifespan of the animal. Moreover, even if a moderate amount of antiserum is collected, this may lose potency with time in storage. Replenishment of a PAL in the form of immortalized lines can thus have significant savings over polyclonal antiserum with respect to reproducibility, time, and quantities of immunogen needed (which could be in limited supply in some circumstances).

An additional novel feature of a PAL is that although it can be a pool or mixture of antibodies that are individually monoclonal in origin, it is not necessary to separately purify or modify the individual monoclonal antibodies comprising the pool, nor is it necessary to clone the individual hybridomas or other cells from which they originate.

A further novel feature is that the libraries can evolve and be adapted to be more selective by adding monoclonal antibodies to the PAL or the corresponding antibody-secreting cells to the producer pool that are different from antibodies of the original PAL but are not directed against specifically desired antigens derived from further rounds of screening.

Methods by which the PALs of this invention are produced can provide significant advantages over conventional polyclonal antiserum. One advantage is that PALs of this invention are substantially comprised of antibody molecules directed toward the various antigens in the immunogen because the antibodies are derived from selected lymphoid organs (e.g., spleen and/or peripheral lymph nodes). Those organs and tissues contain antigen-specific B-lymphocytes that are in a proliferating state after recent immunization. When these proliferating cells are immortalized, they typically continue to produce antibodies to the same antigens that they responded to in the immunogen. Thus, the immortalization process can be relatively selective for specific antibody production. By contrast, specific antibodies to a given immunogen in conventional antiserum generally represent a minority of the total population of antibodies present, rarely reaching more than about 5% to about 10% of the total numbers of immunoglobulin molecules, and mostly are present in even smaller amounts. One technique that has been used to enrich specific antibodies from antiserum is affinity purification. However, affinity purification typically requires a known antigen affixed to an affinity matrix. Affinity purification of specific antibodies in antiserum is difficult to carry out when it is desirable to have a large number of different antibodies directed toward a variety of different, possibly undefined antigens on a scale needed for desired purposes, such as antigen masking.

Another advantage of the PALs of this invention over conventional antibody preparations obtained from antiserum is that PALs can be relatively devoid of extraneous and/or undesired antibodies that are typically found in polyclonal antiserum. Undesired antibodies include high levels of so-called “natural antibodies” with wide-ranging or widely cross-reactive specificities to antigens in the environment, cells from other species, or to intestinal flora of the animal used for producing antiserum. In some cases, these cross-reactive specificities include antigens of mammalian cells, which could be undesirable For example, a significant proportion of natural antibodies or undesired antibodies may be produced by lymphoid tissues associated with the gut or bone marrow, which are not typically harvested for the purpose of making hybridomas. Removal of these undesired antibodies would typically require absorption of antiserum with large amounts of animal tissue, which may be impractical with large fluid volumes and may introduce contaminants into the antiserum. In contrast, PAL of this invention contain antibodies more likely to be directed toward antigens present in the immunogen.

Depending on the chosen range of specificities, a PAL can be used for a variety of purposes, for example, to facilitate the discovery and identification of other monoclonal or polyclonal antibodies specific for antigens associated with a desired normal cell lineage, ontogenetic or maturation level or stage, or activation or functional stage. Polyclonal antibody libraries can also be used to help to identify antibodies specific for antigens on cells associated with disease processes, for example, different types of tumor cells or stages of malignancy, or cells involved in autoimmune, inflammatory, infectious or other disease states or conditions characterized by antigenic expression. In addition, a PAL can be used to help to identify monoclonal antibodies directed to a distinct epitope on a given antigen. Further, a PAL can be used to help to identify monoclonal antibodies having more desirable binding kinetics or higher affinities to any given antigen.

Some additional nonlimiting examples of the use of PALs for such applications include the following: (a) using a PAL made to mature cells in different lineages when screening for cells producing antibodies to antigens on stem cells or precursor cells for those lineages; (b) screening cells producing antibodies to antigens on activated cells or cells involved in autoimmune or inflammatory disease processes e.g., T or B lymphocytes, killer cells, dendritic cells or other antigen-presenting cells (“APC”), regulatory lymphocytes or APC) using a PAL made to the non-activated or normal cell counterparts; (c) using a PAL made to antigens of a less virulent strain of an infectious microorganism, e.g., a bacterium or virus, when screening for antibodies to antigens on other strains associated with greater virulence; and (d) making a PAL to a known antigen in order to screen for cells producing antibodies to a distinct epitope or that bind with higher affinity (i.e., by displacing lower affinity antibodies with similar specificity). It should be apparent to those in the art that monoclonal antibodies whose production is facilitated by the use of PALs in any of these types of nonlimiting examples may have more desirable properties as therapeutic drugs. In addition to facilitating the production of monoclonal antibodies, PALs themselves may have direct utility as therapeutic drugs, diagnostic reagents for diseases, and as reagents to aid in analysis of genomic expression systems or proteome systems.

The production of PALs can employ any method known in the art for raising antibodies to complex mixtures of antigens, including intact cells, cell extracts or fractions. Polyclonal antibody libraries can also be made using phage display antibody methods. The nature of the immunogen can be varied to obtain the desired level of specificity or stringency for the baseline antigen subtraction with the staging libraries. For example, to screen for antibody-producing hybridomas specific for novel antigens on cancer cells, it can be desirable to make PALs with specificities for the normal cell lineage from which the cancer arose. The PALs so made can then used to modify an antigen substrate containing the potential cancer antigens before screening the modified antibody-secreting cells on the substrate. Using prostate cancer as example that is applicable to any other type of tumor, a PAL made against normal prostate tissue can be used when screening for antibody-producing hybridomas to prostate tumor specific antigens. A more stringent example in the cancer field might be to make PALs for staging using primary tumor cells as immunogens and to screen hybridomas made against metastatic cells of the same tumor (or vice-versa).

For any of these purposes, the cells used for the immunization or for producing the antigen substrate, whether as intact cells, cell extracts or fractions, organelles, digests, and the like, can be obtained from commonly available resources and culture techniques, or they can be modified by recombinant techniques. In alternative embodiments, to increase an immune response of an antibody-producing cell, cells of the immunogen can be transformed and cultures expanded to provide a large pool of immunogen. A variety of both normal and malignant cell types are available from commercial suppliers and/or repositories (e.g., American Type Culture Collection or “ATCC”) as primary tissue or established or transformed lines.

Genetic modification of cells for such purposes can involve, for example, upregulating or downregulating a particular gene, or transfecting a host cell line with a single gene or cDNA, a collection of genes or an entire cDNA library such as a subtraction library. For example, suitable host cells for transfection include lines available from the ATCC such as: human cervical carcinoma cells (HELA); human lung cells (W138); human liver cells (Hep G2); human embryonic kidney line (293); monkey kidney cells (CV1); monkey kidney CV1 line transformed by SV40 (COS-7); baby hamster kidney cells (BHK); chinese hamster ovary-cells-DHFR (CHO); african green monkey kidney cells (VERO-76); canine kidney cells (MDCK); buffalo rat liver cells (BRL 3A; mouse mammary tumor (MMT). For these applications the term “host cell” refers to those vertebrate cells capable of growing in culture and expressing desired antigen(s). While the preferred host cells of this invention are vertebrate cells, other eukaryotic cells can be used, such as for example, insect cells. To detect antigens that arise from infection by procaryotic cells (e.g., bacteria) or viruses, PALs can be made to both uninfected target cells and the prokaryotic cell or virus. Thus, a staging library can be prepared that can react with both the normal cellular antigens and pathogen's antigens. Then, testing target cells infected with the bacterial (or virus) can permit detection of cell and/or pathogen-specific antigens produced as a result of the infection.

Techniques for transfecting or transducing genetic material into such lines are described in numerous laboratory methods manuals known in the field of molecular biology. These include, for example: electroporation, calcium phosphate precipitation, liposomal vectors, synthetic vectors, adenoviral vectors, and retroviral vectors.

Alternatively, intact cells can be used as carriers or indicators for exogenously obtained antigens by coupling them with haptens, peptides, soluble proteins or extracts or fractions from other cells. The carrier cells used for this purpose can be nucleated or non-nucleated (e.g., mammalian erythrocytes). A variety of methods are known in the art for accomplishing such couplings, including, for example, direct chemical conjugation, using chemical crosslinkers, or biotin-avidin bridging.

The process for producing a PAL can employ the same initial methods known in the art for making monoclonal antibodies. Using the preferred approach of generating hybridomas, the lymphoid organs of the immunized animals are harvested and fused with parental myeloma cells as referenced above. The hybridomas are then subjected to drug selection (e.g., with standard HAT-containing medium) to eliminate non-fused myeloma cells. However, unlike traditional hybridoma methods where the cells are drug selected following seeding in limiting dilution culture in microwells, the cells for producing staging libraries can be drug selected collectively while they are in batch culture. The cells can then be expanded in culture or in ascites for the purpose of obtaining useful quantities of secreted polyclonal antibodies.

It can be desirable to prevent a reduction in overall specific activity or to increase the activity of relevant antibodies in the culture medium or ascites fluid. Activity can be diminished by overgrowth of nonsecreting hybridoma cells or cells secreting irrelevant immunoglobulin molecules, for example. It is commonly known that overgrowth of undesired populations can be reduced by early expansion of cell lines and cryopreserving in aliquots for future limited-duration use, rather than by continuous serial passaging. In addition, methods are known in the art of cell immunoselection that could be adapted to separate and recover hybridoma cells that retain their surface antigen receptors from those presumptive non-antibody producing cells that do not.

Selecting hybridoma cells as a source of a PAL can be based on the generic presence or amount of secreted antibody product and can be accomplished by techniques such as staining the captured antibody with a complementary fluorescent anti-immunoglobulin antibody and then sorting by flow cytometry. Alternatively, one can select cells by labeling the captured antibody with complementary anti-immunoglobulin antibody linked to immunomagnetic beads and then magnetic cell sorting. It can be noteworthy that such retained cell surface immunoglobulin is considered to represent residual membrane bound receptor antibody from an earlier lymphoblast stage that bears no correlation with the amount of antibody that is secreted from the hybridoma. It can be readily appreciated that any other methods for selecting antibody-producing cells can be used to provide staging libraries.

In another embodiment, the specific activity of a PAL can be increased further, and in certain embodiments, can approach 100 percent antigen-specific antibodies. Increasing specific activity can be accomplished by immobilizing secreted antibodies monoclonally onto the surface membranes of the respective hybridoma or other cells of origin according to the methods of modifying antibody-secreting cells described herein. These modified secreting cells can then be affinity-selected by adhesion to an antigen substrate that is typically of the same or similar antigen composition as the immunogen used to generate the PAL. This type of selection by specificity can be carried out around the same time (i.e., before, during, or after) as the batch HAT (or other drug) selection or a later time of cell culture or expansion.

Once suitable quantities of antibodies for PALs are produced into culture medium or ascites, the antibodies can be collected and manipulated with consideration to the techniques and reagents used to distinguish these antibodies from the secreted antibody products that are captured on the modified antibody-secreting cells that are being screened (i.e., the capturing moiety). Such distinctions can be based on antigenic or structural markers on the immunoglobulins of different animal species, immunoglobulin classes or isotypes, subclasses, allotypes, or sizes using antibodies or other reagents that are commercially available or available as antibody-secreting hybridomas from cell repositories (e.g., ATCC). For example, cells secreting human monoclonal antibodies can be distinguished from a PAL consisting of murine antibodies by use of anti-human immunoglobulin capturing antibodies.

Another embodiment, when PALs and captured secreted antibodies originate from the same animal species, involves the conversion of PALs to antibody fragments and the concurrent use of capturing antibodies or other moieties that bind to the secreted antibodies in their full-length or structurally intact form. Antibody fragments comprise a portion of a full-length antibody, generally the antigen binding or variable region thereof, and lack all or part of the Fc region. Examples of antibody fragments include Fab, Fab′, F(ab′)₂, Fv fragments, single-chain antibody molecules, and single domain antibody molecules. Fc-specific anti-immunoglobulin class antibodies (such as sheep anti-mouse IgG-Fc or goat anti-human IgG-Fc as a non-exclusionary example) or protein A are examples of commercially available reagents that could serve as capturing moieties. Reagents and kits are also available commercially for preparing antibody fragments and purifying them away from intact antibodies (e.g., from Pierce Chemical Company). It can be appreciated that a PAL can comprise IgG, IgE, IgM or any other type of antibody or antibody fragment. Thus, the discussion of IgG producing cells is not intended to be limiting to the scope of this invention.

Desirably, the reaction between the constituent antibodies in a PAL and the antigen substrate is carried out under conditions in which the antibodies saturate or block all of the antigenic sites on the substrate for which they have specificity. Experimental measures used separately or in combination that favor this include raising the total protein concentration of the staging libraries, and thus the concentrations of the individual constituent antibodies. Empirical calculations show that protein concentrations needed to insure this can be easily obtainable.

Other useful measures favoring saturation binding include diluting the antigen concentration on the substrate, lowering the temperature during the interaction to reduce antibody dissociation rate, conducting the monoclonal antibody screening assay in the presence of excess PAL rather than washing it out beforehand, and/or covalently attaching staging libraries to the antigen to prevent dissociation. Covalent attachment offers an advantage in allowing PAL-treated antigen substrates to be prepared hours, days or weeks in advance of antibody-secreting cell screening. A variety of homobifunctional and heterobifunctional chemical crosslinking reagents and kits are available commercially (e.g., from Pierce Chemical Company) that can be adapted for this purpose including, but not limited to those containing reactive aldehydes, succinimidyl esters, imidoesters, maleimidyl esters, hydrazides, aryl azides, and carbodiimides.

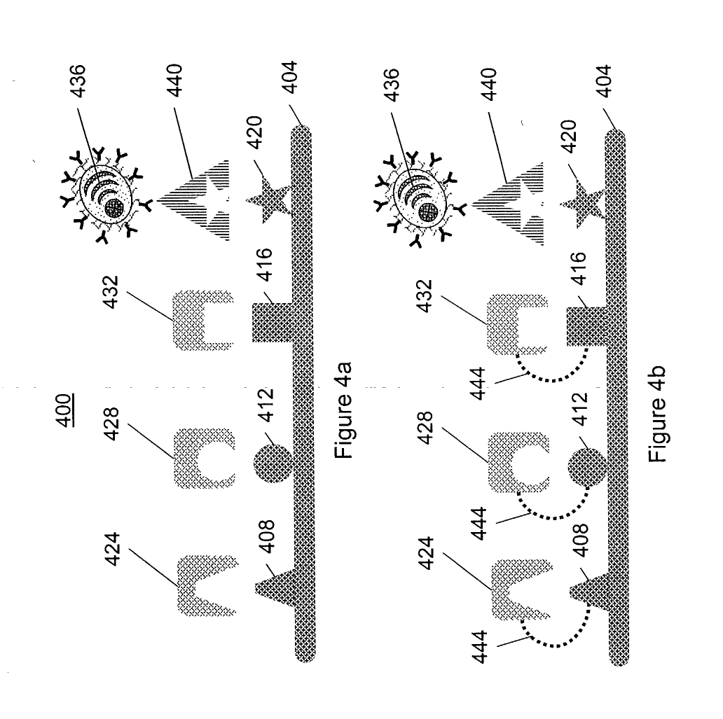

FIGS. 4a and 4b depict schematically, an embodiment of this invention 400 having a substrate 404, with antigens 408, 412, 416 and 420 thereon. Antibodies 424, 428 and 432 in a PAL interact with and mask their respective antigens present in a heterogeneous antigen substrate. Antigen 420 is not masked by antibodies in the PAL, and remains accessible for binding by a modified antibody-secreting cell 436 (not drawn to scale). An enlargement of the binding specificity of captured antibody 440 is depicted for the modified antibody-secreting cell. FIG. 4a, the bound antibodies of the PAL are not covalently attached to their respective antigens, whereas, in FIG. 4b they are covalently attached with cross-linkers 444.

The ability to provide stable PAL-treated antigen substrates can permit the construction of kits suitable for a variety of uses. By way of example only, kits can be provided that comprise PAL-producing cells in frozen form. Expansion of PAL-producing cells can provide a renewal source of staging antibodies for a method for detecting novel antibodies by antigen subtraction using “staging library binding and antigen subtraction” or “SLABS™”. Additionally, a PAL can be in the form of partially or more completely purified antibodies. Such preparations can be obtained from hybridoma cell supernates or ascites. A PAL can be stored in liquid medium, or can be lyophilized to form a more stable powder. Other kit embodiments include substrates comprising a heterogeneous antigen mixture associated therewith “heterogeneous antigen substrates,” and a separate preparation including one or more PALs. Alternatively, a kit can contain one or more heterogeneous antigen substrates with a PAL bound thereto, either covalently or non-covalently. In yet other embodiments, a kit can comprise heterogeneous antigen mixtures derived from a variety of different cell types, including normal cells, diseased cells, or pathogens that can infect or transform normal cells, fragments thereof, and the like.

One or more of the aforementioned methods can find use in the context of screening antibody-secreting cells such as hybridomas. However, it can be readily appreciated by those skilled in the art that methods of this invention can be applied more broadly to any cell type producing an antibody that can be captured on its surface in substantially monoclonal form and capable of binding to an antigen substrate. The origins of such cells can include bacteria, bacteriophage, eucaryotic cells genetically modified to produce antibodies (e.g., transfectomas), or single non-immortalized primary antibody-forming or plaque-forming cells or plasma cells.

As indicated elsewhere in this disclosure, PALs themselves may have direct utility as therapeutic drugs, in addition to facilitating the production of monoclonal antibodies. Polyclonal antibody drugs may have advantages over monoclonal antibodies in cases where, for example, a disease entity is antigenically heterogeneous, prone to mutation, or multi-component in composition. These situations are commonly encountered with cancer, bacterial or viral infections, and exposure to toxins or venoms. Indeed, human immune globulins (e.g., Gamimune from Bayer, Elkhart, Ind.) have been used both prophylactically and for treatment of infections in patients with autoimmune diseases and with transplant-related immunosuppression. Polyclonal anti-human thymocyte antibodies in the form of gamma globulin preparations from rabbits (e.g., Thymoglobulin from Sangstat, Fremont, Calif.) or horses (e.g., Atgam from Pharmacia-Upjohn, Kalamazoo, Mich.) have been used for immunosuppression in human patients for transplant rejection) and autoimmune disorders. Heteroantiserums, gamma globulins and purified polyclonal antibodies from horses and sheep have been used as antivenoms to treat envenomations by species such as snakes, lizards, spiders and insects (Antivenin from Wyeth-Ayerst, Collegeville, Pa.; CroFab from Protherics, Nashville, Tenn.; CSL, Victoria, Australia). Efforts are being made by a number of groups to engineer human immunoglobulin transgenic animals (e.g., mice, rabbits, and cows) capable of producing human monoclonal antibodies or polyclonal antiserums for various types of therapies (e.g., Abgenix, Fremont, Calif.; Medarex, Princeton, N.J.; THP, Mountain View, Calif.; Hematech, Westport, Conn.). The methods of producing both PALs and PAL-falicitated monoclonal antibodies are fully-compatible with such transgenic animals.

The production of therapeutic PALs would be both economically and therapeutically advantageous over polyclonal antibody or gamma globulin preparations from antiserums. Of particular note, a PAL would require substantially less immunogen than a polyclonal antibody preparation from antiserum, since a PAL may be derived from a single immunized animal rather than colonies or herds of animals. Also, unlike a polyclonal antibody preparation from antiserum, preparation of purified antibodies in a PAL would not require antigen for affinity purification. In addition, compared to polyclonal antibody preparation from antiserum a PAL would be more reproducible from batch to batch (being renewable from the same cell source), may have a much higher content of specific antibodies (allowing lower immunoglobulin dosages in patients with less chance of sensitization or anaphylactic reactions), and may be less prone to contamination by adventitious infectious agents in animal products (e.g., bovine spongiform encephalopathy agent responsible for “mad cow” disease). Finally, a therapeutic PAL may be developable as a single drug entity similar to an antiserum, and unlike pools of individually characterized monoclonal antibodies which may be subject to more complicated medical regulatory requirements

The following synopsis of an antigen substrate modification using SLABS is offered as an example and not by way of limitation:

### Example 4

### SLABS Modification of an Antigen Substrate

With the intention of screening for antibody-producing hybridomas to antigens specific for prostate tumor cells, groups of mice are immunized with either normal prostate-derived cells or prostate tumor cells. Hybridomas are generated from the lymphoid organs of the normal cell-immunized mice, HAT selected and expanded collectively in batch culture, and 10-20 liters of conditioned medium were produced. The IgG antibodies are purified by protein A chromatography, digested to Fab′ fragments with pepsin, further purified and concentrated.

Prostate tumor cells are seeded into petri dishes or multiwell plates to establish the antigen substrate. The cells are grown until they reached a state of near-confluency. Alternatively, they are grown elsewhere and then attached using the adhesion promoting chemical polylysine. After removal of nonadherent tumor cells by washing, an aliquot of the SLABS antibody solution is added and incubated for a minimum of one hour. The substrate is then ready for reaction with the modified hybridoma cells raised against prostate tumor antigens.

Alternatively, the SLABS antibodies are covalently crosslinked to their respective antigens on the substrate so that it can be used the following week. The chemical crosslinking agents glutaric dialdehyde (i.e., glutaraldehyde), disuccinimidyl suberate or a water-soluble carbodiimide are used to equal effect. The crosslinker is then washed away and the substrate is stored until needed.

C. Combined Use of the Modified Antibody Antibody-Secreting Cells with the Modified Antigen Substrate

One embodiment for using PALs is in conjunction with the screening of antibody-secreting cells generated against immunogens comprising heterogeneous mixtures of potential antigens. It should be understood that heterogeneous mixtures of potential antigens can be obtained even when a given molecule is defined or cloned, if it is expressed, coupled with or otherwise present among a heterogeneous mixture of other molecules (e.g., intact cells) used for immunization. In preparation for such screening applications, the immunogens used to generate the PALs and the antibody-secreting cells are chosen such that each can contain a range of antigen specificities that overlaps substantially but not completely with the other.

Thus, in accordance with the aforementioned methods, modified antibody-secreting cells can be obtained with captured secreted antibodies on their surfaces in flexible, extended configurations (i.e., FLABS) that are sterically accessible to antigens contained in the substrate. In addition, modified antigen substrates are obtained in which certain categories or subsets of the heterogeneous antigen composition present therein are masked or blocked with antibodies of the PAL, and are thus made inaccessible to binding by the captured antibodies on certain of the modified secreting cells. Combining and reacting these two modified entities results in the selective antigen-specific adhesion of the modified secreting cells to antigens that remain unblocked by PAL antibodies. After removal of the nonadherent cells, the specifically adherent cells can then be recovered and used to produce specific antibodies in any desired quantity as either monoclonal antibodies or pools of antibodies (e.g., PALs).

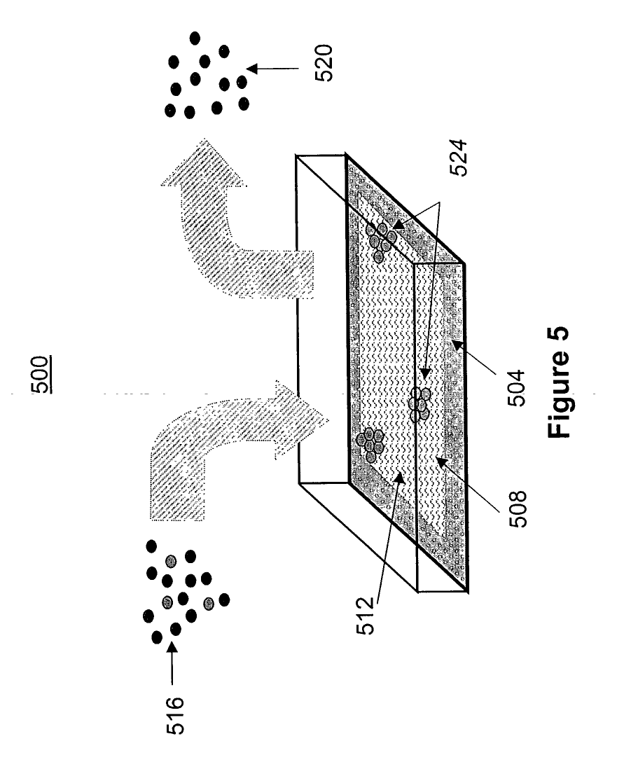

FIG. 5 is a schematic nonlimiting embodiment of an overall selection process 500, involving a dish 504, containing an antigen substrate 508 that is treated with a PAL 512. A suspension of antibody-secreting cells modified with FLABS 516 is brought into contact with the substrate. Nonadherent cells 520 are removed and specifically adherent cells 524 are cultured in situ, forming colonies that are later collected.

In applications where the modified antibody-secreting cells are hybridomas, processes of antigen-specific adhesion can be made more efficient by subjecting the hybridoma cells to drug selection, such as with HAT, for a period of days (typically 2-5 days following cell fusion) before their modification with FLABS. This can substantially reduce the cell numbers (typically by several hundred- to several thousand-fold or greater) that are reacted with the modified antigen substrate, and thus also reduces the size or surface area of the substrate or the amount of materials used for producing the substrate. A ratio of 5 million or fewer drug selected and modified hybridoma cells, or more alternatively 2 million such cells or fewer per cm² of antigen substrate can be effective for specific binding. It is common in the hybridoma field to maintain the hybridomas under drug selection pressure for a total period of up to 7 or 10 days following cell fusion. Thus, the method of culturing following antigen-specific adhesion can usefully employ a continued drug selection for the appropriate total interval.

In another embodiment, an antigen substrate is a horizontal, relatively flat surface such as a dish, plate, membrane, ribbon or film. Adhesion of the modified antibody-secreting cells to specific antigens can then be achieved by settling under unit gravity over a reaction period of 5 minutes to 2 hours. A shorter interval can be more effective with higher concentrations of antigen-specific cells or with a smoother substrate surface containing cell extracts or fractions. A longer interval can be more effective with lower antigen-specific cell concentrations or with an irregular substrate containing intact cells. The interval can be shortened further by applying centrifugal force to the modified secreting cells on the substrate. If the surface is porous, then the interval can also be shortened by filtration of the medium in which the modified secreting cells are suspended through the substrate. Techniques similar to those known in the art for cell “panning” can then be used to remove or wash off the nonadherent cells. These include, for example, inverting the surface, swirling or shaking with liquid medium, flushing or rinsing with additional medium, or aspirating or decanting with any appropriate amount of force or number of repetitions. Such operations can be performed either manually or with automated instrumentation to provide more reproducible or uniform fluid (i.e., hydrodynamic) shear stress, or incremental levels of shear stress. The means to perform and optimize these manipulations are within the skill of those in the art.

In nother embodiments, the surface can be tubular and optionally also porous, such as a capillary tube used for microdialysis or microfiltration. The modified secreting cell suspension can then be brought into contact with the antigen substrate by pumping the suspending medium through the tubes or tube walls, allowing time for the reaction, and then washing off the nonadherent cells by applying a higher tangential fluid shear force.

The adherent modified secreting cells can then be collected from the substrate shortly after selection (i.e., within hours to 1-2 days) by a variety of techniques. For example, when the substrate is relatively flat or horizontal, techniques for aspiration or suction of the cells into a tube or pipette tip can be conveniently aided by high power microscopic visualization of the adherent cells and instrumentation for micromanipulation. Visualization can be based on cell refractive properties, or can employ a viable staining technique such as immunofluorescence microscopy or immunobead labeling using an antibody or avidin that binds to available sites on the anchoring, bridging, or capturing moieties or the captured secreted antibody. Alternatively, the cells can be visualized directly by fluorescence microscopy if fluorescein or some other fluorescing hapten is used as a member of a binding pair in the FLABS anchoring or capturing moieties. The cells can be collected individually in this way for further manipulation.

An alternative approach to collecting and manipulating the adherent secreting cells individually is to collect them as pools of cells that are subsequently aliquotted or sorted as individual cells for further manipulation. This can be an attractive option when large numbers of adherent secreting cells are involved, such as in high-throughput screening operations, because the cells are highly enriched for novel or other desired specificities by virtue of their selection on a SLABS-modified antigen substrate. Collecting the adherent modified secreting cells en masse as pools can be performed by applying a fluid shear force that is sufficient to detach the cells.

The detachment of the adherent modified secreting cells from the antigen substrate as individual or pooled cells can also be facilitated by warming the substrate to physiological temperature and allowing the cells to secrete additional antibody to displace captured secreted antibody bound to the substrate. In addition, warming the substrate to physiological temperature can allow the FLABS to be “capped” or modulated off of the adherent modified secreting cells. If a hapten is used as a member of a binding pair in the FLABS assembly, then partial or complete dissociation of the FLABS also can be achieved by incubating with a solution of the free hapten.

Several options exist for further manipulating the adherent modified secreting cells collected from the antigen substrate shortly after adherence and removal of nonadherent cells. If the collected adherent cells are hybridomas or other cells that have continuous or immortal replicating potential, then they can be cultured elsewhere for further expansion, cloned or subcloned, or subjected to further drug selection such as with HAT, if appropriate, leading to isolation of the desired monoclonal antibodies. Alternatively, mRNAs or genomic DNAs can be extracted from the individual cells in order to molecularly clone and express the antibody van able region cDNAs, as for example described by Lagerkvist et al. (1995) BioTechniques 18:862 and Babcook et al. (1996) Proc. Natl. Acad. Sci. USA 93:7843, the methods of which are herein incorporated by reference. This approach can be preferred when the adherent modified secreting cells are primary AFC which have limited replication or proliferation potential.

If the adherent secreting cells are hybridoma cells or other cells with further proliferation potential, then an alternative to collecting them shortly after adhesion and selection is to culture them in situ directly on the antigen substrate, until such time as they grow to form macroscopic colonies that can be easily visualized by low power microscopy or by the unaided eye. Culturing the cells can be accomplished by overlaying the substrate and adherent cells with a growth medium that contains a low temperature gelling substance such as soft agarose or methylcellulose to prevent cell dispersion. The medium can also include components for further drug selection of the hybridomas if appropriate. The macroscopic colonies that typically appear in 3-10 days can be easily harvested by aspiration or suction. Cells comprising the colonies are largely nonadherent to the antigen substrate because their secreted antibodies saturate their respective antigens on the substrate. The collected adherent cells can then be cultured for further expansion, cloning or subcloning, and selection, as appropriate.

Whereas any of the aforementioned methods for collecting and manipulating the adherent modified secreting cells individually can typically lead to the production of monoclonal antibodies, a variation of these methods can be also used to create the pools or mixtures of antibodies comprising the PALs. For such a purpose the antigen substrate can be used with or without prior contact with another PALs library. In either case, PALs resulting from antigen specific adhesion of the hybridoma cells of origin can then consist entirely or almost entirely of antigen-specific antibodies.

A collection of modified hybridoma cells adhering to a substrate that has been uncontacted by other PALs can constitute a primary PAL with a relatively broad or unrestricted range of antigen specificities. Alternatively, a collection of modified hybridoma cells adhering to a substrate that has been previously contacted by other PALs can constitute a secondary PAL with a relatively narrower or more restricted range of antigen specificities. Thus, it will be evident to those practicing this art that iterative processes of contacting antigen substrates with PAL can be used to produce additional PALs with even narrower or more restricted ranges of specificities. These can be used separately or pooled with other PAL to create tertiary or even higher order PALs which have evolving compositions.

Techniques used for collecting adherent hybridoma cells to generate PALs are generally similar to those used for collecting the cells to generate monoclonal antibodies. Individual adherent cells can be harvested shortly after selection and then pooled. Alternatively, after further growth, individual colonies can be harvested and then pooled. A further alternative option is to harvest the entire population of adherent hybridoma cells collectively or en masse using any of the means described for dislodging the cells. The entire population can then be expanded in culture or in ascites, with or without further selection, as appropriate.

PALs can further promote antigen-specific binding and selection of modified hybridoma cells and other antibody-secreting cells by preventing adhesion molecule-mediated heterotypic cell-cell interactions with other cells that can comprise an antigen substrate. Virtually all of the known individual members of adhesion molecule families (including integrins) have been defined serologically by antibodies, and many of these antibodies are known to block or inhibit the binding properties of these adhesion molecules. Such inhibiting antibodies can be present in the PALs and can be used for adhesion molecule blocking without necessarily having to know the individual specificities or adhesion molecules being blocked. If situations arise where adhesion molecule blocking can not be completely effective, blocking can be augmented by adding monoclonal antibodies with the appropriate defined specificities. Alternatively, small molecule inhibitors or soluble molecular forms of one or both members of an adhesion ligand-receptor binding pair can be added.

Once the recovered modified secreting cells are allowed to grow and secrete larger amounts of antibodies, the capturing antibodies contained in the FLABS are unlikely to have a significant effect on secretion because concentrations of secreted antibodies will rapidly exceed the binding capacity of the capturing antibodies. In addition, as the cells divide, the capturing antibodies are substantially diluted on the cell surface.

Methods provide for performing a reselection or secondary selection of the recovered modified secreting cells on a similar or a different antigen substrate or a substrate that has been reacted with the same or a different PAL antibody library. It can be beneficial in such situations to more rapidly strip components of the FLABS from the cells involved in the adherence to the previous antigen substrate, including the captured secreted antibody or the capturing or bridging moieties. When the FLABS assembly includes a member of a binding pair such as a hapten (e.g., fluorescein, biotin, a nitrophenol derivative, or digoxigenin or any analogues or derivatives thereof), then incubation of the cells with a high molar excess of the same hapten in soluble form can result in dissociation of the assembly. A bridging moiety that is digestible with an enzyme or that has a binding partner that is digestible can be used to similar effect.

Methods also provide for measures to preclude the possibility of contamination of the recovered modified secreting cells or of the secreted antibodies by intact viable target cells, other extracted material or potentially infectious agents derived from the antigen substrate. Treatment of the antigen substrate with crosslinking agents used to immobilize the PALs antibodies according to the aforementioned methods can be effective in such situations. Other measures to prevent growth of intact cells or emergence of infectious agents, particularly when crosslinkers are not used, include treating the substrate (prior to reaction with the hybridoma cells) with an inhibitor of DNA replication or growth such as irradiation, mitomycin C, or psoralen compounds. Protocols for treating cells by such means are known to those skilled in the art.

### Example 5

### Generation of SLABS Antibodies for Normal Prostate Antigens from Hybridomas

To obtain monoclonal antibodies specific for novel antigens on human prostate tumor cells, mice were immunized with normal human prostate-derived cells or human prostate tumor cells. Hybridomas were generated from the lymphoid organs of the normal cell-immunized mice, cultured in bulk with HAT selection for 3-5 days, and the surviving hybridoma cells were then modified with FLABS according to the aforementioned methods and examples (section A above) to capture the secreted antibody.

In accordance with the aforementioned methods and examples for modification of antigen substrates (section B above), normal prostate cells were seeded into petri dishes or multiwell plates to establish the antigen substrate. The cells were grown until they reached a state of near-confluency. Alternatively, they were grown elsewhere and then attached using the adhesion promoting chemical polylysine. The antigen substrates were then irradiated (10 Gy) or treated with a solution of mitomycin C for an hour and then washed extensively to remove excess reagent.

The FLABS-modified hybridoma cells were adjusted to a suitable concentration, incubated with the antigen substrate for 1 hour in the cold, and nonadherent cells were then washed off. The substrate with adherent hybridoma cells was overlaid with cell culture medium containing a low temperature (ca. 37-40° C.) gelling agarose and HAT, and cultured for a further 5-7 days. The macroscopic hybridoma colonies were then collected by aspiration, pooled, and expanded in batch culture. The IgG antibodies were purified from the conditioned medium, digested to Fab′ fragments with pepsin, and concentrated. PALs antibodies to normal prostate antigens were thus obtained.

### Example 6

### Modification of Prostate Tumor Cell Antigen Substrate with PALs

Prostate tumor cells were seeded into petri dishes or multiwell plates to establish the antigen substrate. The cells were grown until they reached a state of near-confluency. Alternatively, they were grown elsewhere and then attached using the adhesion promoting chemical polylysine. The antigen substrates were then irradiated (10 Gy) or treated with a solution of mitomycin C for an hour and then washed extensively to remove excess reagent.

An aliquot of the PALs antibody solution made against normal prostate antigens was added to the tumor cell antigen substrate and incubated for a minimum of one hour. The substrate was then ready for reaction with the modified hybridoma cells raised against prostate tumor antigens.

Alternatively, the bound PALs antibodies were covalently crosslinked to their respective antigens on the substrate so that it could be used later. The chemical crosslinking agents glutaric dialdehyde (i.e., glutaraldehyde), disuccinimidyl suberate or a water-soluble carbodnmide were used to equal effect. The excess crosslinker was then washed away and the substrate was stored until needed.

### Example 7

### Adhesion and Selection of Prostate Tumor Antigen-Specific Hybridomas for Monoclonal Antibody Production

Hybridomas were generated from the lymphoid organs of the prostate tumor cell-immunized mice, cultured in bulk with HAT selection for 3-5 days, and the surviving hybridoma cells were then similarly modified with FLABS according to the aforementioned methods and examples (section A above) to capture the secreted antibody.

FLABS-modified hybridoma cells were adjusted to a suitable concentration, and then incubated with the PALs-modified prostate tumor antigen substrate (described in example 6) for 1 hour in the cold. After washing off nonadherent cells, the substrate with adherent hybridoma cells was overlaid with cell culture medium containing a low temperature (ca. 37° C.) gelling agarose and HAT, and cultured for a further 5-7 days. The macroscopic hybridoma colonies were then collected individually by aspiration, and either expanded in culture directly or subcloned by limiting dilution culture and then expanded. The IgG antibodies were then purified from the conditioned medium and used appropriately.

### Example 8

### Adhesion and Selection of Prostate Tumor Antigen-Specific AFC for Monoclonal Antibody Production

A fraction of cells substantially enriched in antibody-forming cells (AFC) was obtained by velocity sedimentation of spleen or lymph node cells through a low density medium at unit gravity, or through a density gradient at low centrifugal force from mice immunized repeatedly to human prostate tumor cells. The enriched cells were then treated in the same manner as in the aforementioned third example to generate the FLABS anchoring, bridging and capturing moieties.

FLABS-modified AFC were adjusted to a suitable concentration, and then incubated with the PALs-modified prostate tumor antigen substrate (described in example 6) for 1 hour in the cold. After washing off nonadherent cells, the adherent AFC were visualized by direct fluorescence microscopy by virtue of the fluorescein hapten comprising member of a binding pair in the FLABS assembly. Individual fluorescent cells were then collected by micromanipulation. Techniques for mRNA extraction from each isolated cell and for molecular cloning and expression of the antibody heavy and light chain genes were similar to those described by Babcook et al. (1996) ibid.

Alternatively, the adherent cells were collected as a pool by more forceful flushing of the substrate after incubation of the substrate at physiological temperature to permit additional antibody secretion. After determining the cell concentration, aliquots from the pool containing individual cells were then made and mRNA was extracted from each isolated cell for molecular cloning and expression of the antibody heavy and light chain genes.

### Example 9

### Preparation of a Rabbit Hybridoma-Derived Polyclonal Antibody Library (PAL) Against Human T Cells for Immunosuppression Therapy

Therapeutic PALs useful for patient immunosuppression in transplant recipients or patients with autoimmune disorders are generated against antigens on proliferating human T cells. Normal laboratory rabbits (e.g., New Zealand white) or human immunoglobulin transgenic rabbits (e.g., from THP, Mountain View, Calif.) are immunized with thymocytes of surgically resected human thymus glands or cells of a cultured human T lymphoblastoid line (e.g., Jurkat, available from ATCC). Hybridomas are generated from the lymphoid organs of these rabbits by fusion with either a standard mouse drug (HAT) sensitive myeloma partner (producing heterohybridomas or heteromyelomas) or a rabbit myeloma partner (Spieker-Polet H, et al., Proc Natl Acad Sci USA (1995) 92: 9348). The cells are cultured in bulk with HAT selection for 3-5 days, and the surviving hybridomas are modified with FLABS according to the aforementioned methods and examples to capture the secreted antibody.

A T cell antigen substrate consisting of the same types of cells used for the immunization is prepared by seeding the cells at a density suitable to produce a monolayer in a culture dish and attaching them using the adhesion promoting chemical polylysine. The substrate is then irradiated (10 Gy) or treated with a solution of mitomycin C for an hour and then washed extensively to remove excess reagent. The FLABS-modified hybridomas are adjusted to a suitable concentration, and then selected on the antigen substrate according to the aforementioned methods and examples. After removal of nonadherent hybridomas, the adherent antigen-specific hybridomas are collected from the substrate as a pool of cells, and expanded in bulk cell culture (including another 5-7 days of HAT selection). The specific antibodies comprising the PAL are then purified from the conditioned cultured medium by protein A/G chromatography and other standard methods. This selection process can be repeated at any later stage of process development to further reinforce the stability and antigen specificity of the individual hybridomas in the pool.

To optionally restrict further the antigen specificity of the above anti-T cell PAL, selections of the above FLABS-modified hybridomas can be performed on a T cell antigen substrate that is previously treated with another PAL made against non-T cell associated leucocyte antigens. A non-T cell PAL can be made by immunizing rabbits with human peripheral blood leukocytes from donor leukapheresis that have been depleted of T cells. T cell depletion is performed by reacting cells with an anti-T cell monoclonal antibody (e.g., anti-CD2 or anti-CD3) coupled to magnetic beads and passing through a magnetic selection device (e.g., Miltenyi Biotec, Auburn, Calif.) according to manufacturer's directions. Alternatively, human B lymphoblastoid (e.g., IM-9 from ATCC) or myeloid (e.g., HL-60 from ATCC) cell lines can be used. Hybridomas can then be generated from these rabbits, modified with FLABS, selected on non-T cell antigen substrates and expanded in bulk culture as described previously in this example. IgG antibodies are purified from the conditioned medium, digested to F(ab′)2 fragments with pepsin, and concentrated. This PAL is then used to pre-treat the above T cell antigen substrate before selection of the anti-T cell FLABS-modified hybridomas.

### Example 10

### Preparation of a Transgenic Human Immunoglobulin PAL Specific for HIV-Infected T Cells

Methods for preparation of a transgenic human immunoglobulin PAL specific for HIV-infected T cells are similar to those described in Example 9, with certain modifications as indicated. Immunizations and hybridoma production are performed using human immunoglobulin transgenic mice (available from Abgenix or Medarex) in place of laboratory or transgenic rabbits in the previous example. Hybridomas are generated by fusion of immune cells with a HAT-sensitive myeloma cell line. HIV-infected T cells used for both immunization and for selection of antigen-specific FLABS-modified hybridomas are produced by infecting a T lymphoblastoid cell line (e.g., Jurkat clone E-6 or CEM cells available from ATCC) with HIV, such as a T-tropic laboratory strain of HIV, NL4-3 (available from NIH AIDS Research and Reference Reagent Program) or a primary isolate, for approximately 48 hours. The cells are then treated with a psoralen reagent (Sigma Chemicals, St. Louis, Mo.) or other inhibitor of viral replication before use as immunogen or attachment to the antigen substrate.

Other mice are also immunized and hybridomas produced against T cells not infected with HIV. FLABS-modified hybridomas from the non-HIV infected T cell immunized mice are antigen-selected on a non-infected T cell antigen substrate, and then used to produce an anti-T cell PAL. Similar to the preceding example, this PAL is used to pre-treat the HIV infected T cell antigen substrate before selection of FLABS-modified hybridomas made against infected T cells. Adherent hybridomas are collected from the infected antigen substrate and expanded in bulk culture. The specific antibodies comprising the PAL are then purified from the conditioned cultured medium by protein A/G chromatography and other standard methods. Single hybridoma cells may also be optionally subcloned from the cultures in order to obtain monoclonal antibodies. Similar methods for producing PALs could also be applied to other viruses, such as smallpox virus.

### Example 11

### Preparation of a Transgenic Human Immunoglobulin PAL Specific for Bacillus Anthracis (Anthrax Agent)

Human immunoglobulin transgenic mice (Abgenix, Medarex) are immunized with a standard preparation of anthrax vaccine (BioPort, Lansing, Mich.) or other sterilized preparation of Bacillus anthracis, and hybridomas are generated by fusion with a standard mouse drug (HAT) sensitive myeloma partner. The cells are cultured in bulk with HAT selection for 3-5 days, and the surviving hybridomas are modified with FLABS according to the aforementioned methods and examples to capture the secreted antibody. An antigen substrate is produced by adsorbing or chemically crosslinking the vaccine mixture (after dialyzing into suitable buffer) onto a tissue culture dish (e.g., coating the dish with polylysine and crosslinking with glutraldehyde), and antigen-specific FLABS-modified hybridomas are selected. Any non-specific interactions that may occur between bacterial cell wall components and normal hybridoma surface membrane glycoproteins (attributable to bacterial lectins) are precluded by the FLABS structure, which acts as an insulator. Adherent hybridomas are collected from the antigen substrate and expanded in bulk culture. The specific antibodies comprising the PAL are then purified from the conditioned cultured medium by protein A/G chromatography and other standard methods, and can be used as diagnostic or therapeutic reagents.

### Example 12

### Preparation of a Rabbit Hybridoma-Derived Polyclonal Antibody Library (PAL) Against Snake Venom Toxins

A single PAL may be effective as an antivenom for neutralizing the multitude (commonly believed to comprise several dozen or more) of enzymes, toxins and other proteins produced by individual species or entire families of venomous snakes (e.g., Crotalidae). Pooled venoms are used for immunization of normal laboratory rabbits (e.g., New Zealand white) or human immunoglobulin transgenic rabbits (e.g., from THP, Mountain View, Calif.), either at sublethal dosages or after inactivation (e.g., by known methods of heat treatment or formalin fixation). Hyridomas are generated from the lymphoid organs of the rabbits by fusion with either a standard mouse drug (HAT) sensitive myeloma partner (producing heterohybridomas or heteromyelomas) or a rabbit myeloma partner (Spieker-Polet H, et al., Proc Natl Acad Sci USA (1995) 92: 9348). The cells are cultured in bulk with HAT selection for 3-5 days, and the surviving hybridomas are modified with FLABS according to the aforementioned methods and examples to capture the secreted antibody. An antigen substrate is produced by adsorbing or chemically crosslinking the venom protein mixture onto a tissue culture dish (e.g., coating the dish with polylysine and crosslinking with glutraldehyde), and antigen-specific FLABS-modified hybridomas are selected. Adherent hybridomas are collected from the antigen substrate and expanded in bulk culture. The specific antibodies comprising the PAL are then purified from the conditioned cultured medium by protein A/G chromatography and other standard methods.

1. A method of selecting an antibody-producing cell, comprising the steps of:
(a) providing at least one cell producing at least a first antibody not present in a polyclonal antibody library;
(b) associating at least one capture moiety with said at least one antibody-producing cell, said capture moiety associated with a non-antigen recognition portion of said at least first antibody, producing a modified antibody-producing cell;
(c) providing a masked antigen substrate (AS), con-rig an AS pre-treated with a polyclonal antibody library (PAL) to mask antigens on said AS recognized by antibodies of said PAL;
(d) adhering said modified antibody-producing cell to said masked AS by way of said at least fist antibody; and
(e) removing nonadhering cells.

2. The method of claim 1, further comprising the steps of:
(f) collecting the adhering modified antibody-producing cell from the HAS; and
(g) growing the collected modified antibody-producing cell to obtain antibodies.

3. The method of claim 1, wherein step (d) results in antigen-specific binding and adhesion of said modified cell to said AS.

4. The method of claim 1, wherein said AS is a substantially homogeneous antigen substrate.

5. The method of claim 1, wherein said AS is a heterogeneous antigen substrate (HAS).

6. The method of claim 5, wherein said HAS comprises more than one antigen selected from the group consisting of intact cells, cell extracts, cellular organelles, cell fractions and cellular digests.

7. The method of claim 1, wherein said AS comprises antigens derived from a eucaryotic organism.

8. The method of claim 1, wherein said AS comprises antigens derived from a procaryotic organism.

9. The method of claim 1, wherein the said antibody-producing cell is a hybridoma cell.

10. The method of claim 1, wherein the said antibody-producing cell is produced by transfecting a cell with nucleotide sequences encoding said antibody.

11. The method of claim 1, wherein the said antibody-producing cell is an antibody-forming cell (AFC) or a plaque-forming cell (PFC).

12. The method of claim 1, wherein said antibody producing cell is selected from the group consisting of mice, rats, hamsters, humans, monkeys, human/mouse chimeras, human/rat chimeras, and human/monkey chimeras.

13. The method of claim 1, wherein said antibody-producing cell is derived from a bacterium or a bacteriophage.

14. The method of claim 9, wherein said hybridoma cell is grown in a mixed culture not subjected to single cell cloning before contacting with said antigen substrate.

15. The method of claim 10, wherein said antibody-producing cell is grown in a mixed culture not subjected to single cell cloning before contacting with said antigen substrate.

16. The method of claim 9, wherein said cell is grown in a mixed culture and is subjected to drug selection before contacting with said AS.

17. The method of claim 10, wherein said cell is grown in a mixed culture and is subjected to drug selection before contacting with said AS.

18. The method of claim 1, wherein said cell is grown in a mixed culture and subjected to drug selection before contacting with said AS.

19. The method of claim 16, wherein said drug is HAT.

20. The method of claim 17, wherein said drug is HAT.

21. The method of claim 18, wherein said drug is HAT.

22. The method of claim 9, wherein said hybridoma cell is subjected to drug selection after contacting with said antigen substrate.

23. The method of claim 22, wherein said drug is HAT.

24. The method of claim 10, wherein said antibody-producing cell is subjected to drug selection after contacting with said antigen.

25. The method of claim 24, wherein said drug is HAT.

26. The method of claim 11, wherein said antibody-producing cell is subjected to drug selection after contacting with said antigen.

27. The method of claim 26, wherein said drug is HAT.

28. The method of claim 9, wherein said hybridoma cell is removed from said AS and cultured to produce a population of cells that produce antibodies having substantially homogeneous specificities.

29. The method of claim 10, wherein said transfected cell is removed from said AS and cultured to produce a population of cells that produce antibodies having substantially homogeneous specificities.

30. The method of claim 11, wherein said antibody-producing cell is removed from said AS and cultured to produce a population of cells that produce antibodies having substantially homogeneous specificities.

31. The method of claim 28, wherein a plurality of said hybridoma cells is collected as a pool of cells.

32. The method of claim 31, wherein said pool of cells have mixed antigenic specificites.

33. The method of claim 10, wherein a plurality of said transfected cell is collected as a pool of cells having mixed antigenic specificities.

34. The method of claim 11, wherein a plurality of said cells are collected as a pool of cells having mixed antigenic specificities.

35. The method of claim 28, wherein said hybridoma cell is cultured in situ and forms a discrete cell colony.

36. The method of claim 29, wherein said transfected cell is cultured in situ and forms a discrete cell colony.

37. The method of claim 16, wherein the collected adherent hybridoma cells are used to produce monoclonal antibodies.

38. The method of claim 9, wherein a plurality of said hybridoma cells are used to produce polyclonal antibodies.

39. The methods of claim 17, wherein said transfected cell is used to produce monoclonal antibodies.

40. The method of claim 27, wherein said transfected cell colonies are used to produce monoclonal antibodies.

41. The method of claim 2, further comprising the step of extracting mRNA or genomic DNA from said antibody-producing cell.

42. The method of claim 41, further comprising inserting said mRNA or said genomic DNA into a recipient cell and culturing said cell to produce a plurality of cells that express said nucleic acid sequence.

43. The method of claim 10, wherein said nucleic acid sequence lacks a sequence necessary for said antibody to be secreted by said antibody-producing cell.

44. The method of claim 28, further comprising the step of isolating mRNA encoding said antibody.

45. The method of claim 44, wherein said mRNA is used to make a cDNA encoding said antibody.

46. The method of claim 29, further comprising the step of isolating mRNA encoding said antibody.

47. The method of claim 46, wherein said mRNA is used to make a cDNA encoding said antibody.

48. The method of claim 30, further comprising the step of isolating mRNA encoding said antibody.

49. The method of claim 48, wherein said mRNA is used to make a cDNA encoding said antibody.

50. The method of claim 1, wherein step (b) is carried out in a medium that slows diffusion of said antibody.

51. The method of claim 50, wherein said medium comprises a substance selected from the group consisting of low-melting point agarose, gelatin methylcellulose, alginate, percoll, ficoll, albumin and sucrose.

52. An isolated antibody-producing cell, comprising;
a capture moiety associated with a surface of said cell; and
an antibody produced by said cell attached to said capture moiety, said antibody having specificities not present in a PAL used to mask an antigen substrate.

53. A population of antibody-producing cells that adhere to a PAL-treated antigen substrate.

54. A monoclonal antibody produced by an antibody-producing cell that adheres to a PAL-treated antigen substrate.

55. The cell of claim 52, wherein said cell is a hybridoma cell.

56. The cell of claim 52, wherein said cell is transfected with a nucleic acid sequence that encodes said antibody.

57. The cell of claim 52, wherein said cell is an AFC or a PFC.

58. The cell of claim 52, wherein said cell is selected from the group consisting of murine, rat, hamster, monkey, human, human/murine chimeras, human/rat chimeras, human/monkey chimeras and human/hamster chimeras.

59. The method of claim 1, wherein said PAL is manufactured comprising the steps of:
(a) immunizing an animal with immunogen comprising a mixture of antigens not having an antigen of interest;
(b) obtaining a mixture of antibody producing cells from said animal, said antibody producing cells producing antibodies directed toward at least a portion of said antigens from said immunogen; and
(c) immortalizing said antibody producing cells.

60. The method of claim 59, wherein said immunogen comprises antigens selected from the group consisting of cells, transfected cells, cell fragments, cellular ogranelles, cell fractions, cellular digests, cellular molecules and molecular digests.

61. The method of claim 59, where said step of immortalizing is carried out in vitro.

62. The method of claim 61, wherein said step of immortalizing includes producing hybridomas.

63. The method of claim 62, wherein said hybridomas are subjected to drug selection.

64. The method of claim 63, wherein said drug selection is carried out using HAT.

65. The method of claim 59, further comprising the step of collecting said antibodies, forming a polyclonal antibody library (PAL).

66. The method of claim 65, wherein said antibody library contains at least one functional antibody moiety selected from the group consisting of antibody fragments, Fab, Fab′, F(ab′)₂, Fv fragments, single-chain and/or single domain antibody molecules,

67. The method of claim 66, wherein said function is determined by binding of said antibody to an antigen.

68. The method of claim 59, wherein said polyclonal library-producing cells are produced by transfection of antibody genes into another cell line.

69. The method of claim 59, wherein said polyclonal library-producing cells are produced from phage antibody libraries.

70. The method of claim 59, wherein at least one of said antigens is from a eucaryotic cell.

71. The method of claim 59, wherein at least one of said antigens is from a procaryotic cell.

72. The method of claim 59, wherein at least one of said antigens is from a virus.

73. The method of claim 70, wherein said eucaryotic cell is derived from at least one origin selected from the group consisting of ectodermal, endodermal and mesodermal origin.

74. The method of claim 59, wherein at least one of said antigens is selected from the group consisting of primary cells, cell lines and immortalized cells that retain a normal cell antigen phenotype.

75. The method of claim 59, wherein at least one of said antigens is from a normal cell selected from the group consisting of breast, ovary, prostate, colon, rectum, lung, brain, kidney, pancreas, skin, connective tissue, intestinal, muscle, or hematologic cells.

76. The method of claim 59, wherein at least one of said antigens is from a tumor cell.

77. The method of claim 76, wherein said cell is selected from the group consisting of a metastatic tumor cell and a primary tumor cell.

78. The method of claim 59, wherein at least one of said antigens is from a cell is selected from the group consisting of normal mature cells and cells from tissues of a known lineage.

79. The method of claim 78, wherein said cell is a lymphocyte selected from the group consisting of immature lymphocytes, mature lymphocytes and differentiated lymphocytes.

80. The method of claim 78, wherein said lymphocyte is not activated.

81. The method of claim 78, wherein said lymphocyte is selected from the group consisting of T-lymphocytes and B-lymphocytes, killer cells and regulatory cells.

82. The method of claim 59, wherein at least two of said antibodies react to different epitopes of the same antigen molecule.

83. The method of claim 59, wherein said PAL comprises a plurality of monoclonal antibodies derived from a renewable source, at least one of said antibodies not directed toward an antigen of pre-defined specificity.

84. The method of claim 83, wherein said renewable source is an immortalized cell culture.

85. The method of claim 83, which comprises the collected, purified or concentrated secreted antibodies.

86. The method of claim 83, wherein said antibodies are selected from the group consisting of IgG, IgM, IgA, IgD and IgE antibodies.

87. The method of claim 83, wherein said PAL comprises antibody fragments, including Fab, Fab′, F(ab′)₂, Fv fragments, single-chain antibody molecules and single-domain antibody molecules.

88. The method of claim 83, wherein said renewable source comprises immortalized hybridoma cells.

89. The method of claim 88, wherein said hybridoma cells are subjected to drug selection and maintained as batch cultures.

90. The method of claim 83, wherein said renewable source comprises cell lines transfected with antibody genes.

91. The method of claim 90, wherein said renewable source comprises cell lines transfected with phage antibody libraries.

92. The method of claim 1, wherein said PAL is manufactured comprising the steps of:
(a) immunizing an animal with an immunogen comprising a mixture of antigens not having an antigen of interest;
(b) obtaining a mixture of antibody producing cells from said animal, said antibody producing cells producing antibodies directed toward at least a portion of said antigens from said immunogen;
(c) immortalizing said antibody producing cells; and
(d) collecting said antibodies.

93. The method of claim 59, wherein said PAL comprises at least one antigen recognition moiety selected from the group consisting of Fab, Fab′, F(ab′)₂, Fv fragments, single domain antibodies and single-chain antibody molecules.

94. The method of claim 1, wherein the antigen substrate consists of a cellular “lawn.”

95. The method of claim 1, wherein the antigen substrate is organized on a grid allowing each position on the grid to be determined or placed into registry with the modified antibody-secreting cells that are brought into contact.

96. The method of claim 1, wherein the cellular antigen substrate is eucaryotic.

97. The method of claim 1, wherein the cellular antigen substrate is procaryotic.

98. The method of claim 1, wherein said antibody-producing cell is made against a tumor selected from a tissue selected from the group consisting of breast, ovary, prostate, colon, rectum, lung, brain, kidney, and a hematologic cell, and said PAL made against a normal cell or tissue counterpart to the tumor used to produce said antibody.

99. The method of claim 1, wherein said antibody-producing cell is made against a metastasized tumor tissue and said PAL is made against a primary tumor tissue corresponding to said metastasized tumor.

100. The method of claim 1, wherein said antibody-producing cell is made against a primary tumor tissue and said PAL is made against a metatasized tumor tissue corresponding to said primary tumor.

101. The method of claim 1, wherein said PAL is made against a normal cell and the said antibody-secreting cell is made against a precursor cells of said normal cell.

102. The method of claim 1, wherein said antibody-secreting cell is made to an antigen on an activated immune system cell, and said PAL is made to a non-activated counterpart of said immune system cell.

103. The method of claim 1, wherein the said antibody-secreting cell and said PAL react against different epitopes of the same antigen.

104. The method of claim 1, wherein said AS in step (c) comprises antigens from at least one of intact cells, cell extracts, cellular organelles, cell fractions and cellular digests.

105. The method of claim 1, wherein said capture moiety is associated with said antibody-producing cell by way of an anchoring moiety.

106. The method of claim 105, wherein said anchoring moiety is associated with said cell by way of a mechanism selected from the group consisting of antigen recognition, carbohydrate recognition, ionic interaction and hydrophobic interaction.

107. The method of claim 106, wherein said antigenic recongintion mechanism uses an antigen selected from the group consisting of MHC antigens, and cluster of differentiation (CD) antigens.

108. The method of claim 106, wherein said carbohydrate recognition mechanism uses a carbohydrate selected from the group consisting of glycoproteins, glycolipids, and lectins.

109. The method of claim 106, wherein said ionic interaction uses a polycation.

110. The method of 109, wherein said polycation is selected from the group consisting of polylysine, protamine and chitosan

111. The method of claim 1, wherein said capture moiety is an antibody.

112. The method of claim 105, wherein said anchoring moiety is biotin bound to avidin or biotin bound to streptavidin.

113. The method of claim 105, wherein said anchoring moiety is NTA bound to a polyhistidine peptid.

114. The method of claim 105, wherein said capture moiety is associated with said anchor moiety by way of a bridging moiety.

115. The method of claim 114, wherein said bridging moiety is selected from the group consisting of branched polymers, dextrans, polyethylene glycol, polypropylene glycol, polyvinyl alcohol, polyvinylpyrollidone, spheriodal beads and dendrimers.

116. The method of claim 78, wherein said cell is selected from the group consisting of antigen-presenting cells, monocytes, macrophages, dendritic cells, mast cells, granulocytes and regulatory cells.

117. The method of claim 1, wherein said PAL is directed towards a first tumor and said first antibody is directed towards an antigen from a second tumor.

118. The method of claim 105, wherein said anchoring moiety comprises a flag peptide and a flag-peptide specific antibody.

119. A device for selecting an antibody-producing cell, comprising:
a PAL-treated antigen substrate;
means for contacting an antibody-producing cell with said PAL; and
means for removing non-adhering cells from said substrate.

120. A device for growing selected antibody-producing cells, comprising:
a PAL-treated antigen substrate;
means for contacting an antibody-producing cell with said PAL
means for removing non-adhering cells from said substrate; and
means for contacting growth medium with said adherent cells;
